SEQUENCE LISTING (125 sequences)

SEQ ID NO: 1 - protein, 647 aa, Bordetella pertussis
MHIYGNMNRATPCRGAVRALALALLGAGMWTLSPPSAWALKLPSLLTDDELKLVLPTGMSLEDFKRSLQESAPSALATPPSSSPPVAKPGPGSVAEAPSGSGHKDNPSPPVVGVGPGMAESSGGHNPGVGGGTHENGLPGIGKVGGSAPGPDTSTGSGPDAGMASGAGSTSPGASGGAGKDAMPPSEGERPDSGMSDSGRGGESSAGGLNPDGAGKPPREEGEPGSKSPADGGQDGPPPPRDGGDADPQPPRDDGNGEQQPPKGGGDEGQRPPPAAGNGGNGGNGNAQLPERGDDAGPKPPEGEGGDEGPQPPQGGGEQDAPEVPPVAPAPPAGNGVYDPGTHTLTTPASAAVSLASSSHGVWQAEMNALSKRMGELRLTPVAGGVWGRAFGRRQDVDNRVSREFRQTISGFELGADTALPVADGRWHVGAVAGYTNGRIKFDRGGTGDDDSVHVGAYATYIEDGGFYMDGIVRVSRIRHAFKVDDAKGRRVRGQYRGNGVGASLELGKRFTWPGAWYVEPQLEVAAFHAQGADYTASNGLRIKDDGTNSMLGRLGLHVGRQFDLGDGRVVQPYMKLSWVQEFDGKGTVRTNDIRHKVRLDGGRTELAVGVASQLGKHGSLFGSYEYAKGSRQTMPWTFHVGYRYAW

SEQ ID NO: 2 - protein, 12 aa, synthetic construct
  REGION 1..12: note = peptide epitope
PGIGKVGGSAPG

SEQ ID NO: 3 - protein, 12 aa, synthetic construct
  REGION 1..12: note = peptide epitope
APGPDTSTGSGP

SEQ ID NO: 4 - protein, 6 aa, synthetic construct
  REGION 1..6: note = peptide epitope
PDTSTG

SEQ ID NO: 5 - protein, 9 aa, synthetic construct
  REGION 1..9: note = peptide epitope
PDTSTGSGP

SEQ ID NO: 6 - protein, 12 aa, synthetic construct
  REGION 1..12: note = peptide epitope
PADGGQDGPPPP

SEQ ID NO: 7 - protein, 12 aa, synthetic construct
  REGION 1..12: note = peptide epitope
LPERGDDAGPKP

SEQ ID NO: 8 - protein, 9 aa, synthetic construct
  REGION 1..9: note = peptide epitope
EGGDEGPQP

SEQ ID NO: 9 - protein, 36 aa, synthetic construct
  REGION 1..36: note = peptide epitope
NAQLPERGDDAGPKPPEGEGGDEGPQPPQGGGEQDA

SEQ ID NO: 10 - protein, 36 aa, synthetic construct
  REGION 1..36: note = peptide epitope
LPERGDDAGPKPPEGEGGDEGPQPPQGGGEQDAPEV

SEQ ID NO: 11 - protein, 6 aa, synthetic construct
  REGION 1..6: note = peptide epitope
LPERGD

SEQ ID NO: 12 - protein, 9 aa, synthetic construct
  REGION 1..9: note = peptide epitope
RGDDAGPKP

SEQ ID NO: 13 - protein, 9 aa, synthetic construct
  REGION 1..9: note = peptide epitope
DEGPQPPQG

SEQ ID NO: 14 - protein, 6 aa, synthetic construct
  REGION 1..6: note = peptide epitope
PQPPQG

SEQ ID NO: 15 - protein, 9 aa, synthetic construct
  REGION 1..9: note = peptide epitope
PQGGGEQDA

SEQ ID NO: 16 - protein, 9 aa, synthetic construct
  REGION 1..9: note = peptide epitope
PEVPPVAPA

SEQ ID NO: 17 - protein, 9 aa, synthetic construct
  REGION 1..9: note = peptide epitope
VYDPGTHTL

SEQ ID NO: 18 - DNA, 408 bp, synthetic construct
  misc_feature 1..408: note = 14D12 heavy chain
  source 1..408: mol_type = other DNA
atgaacttcgggctcagcttgattttccttgtccttattttaaaaggtgtccagtgtgaagtgatgctggtggagtctgggggagccttagtgaagcctggagggtccctgaaactctcctgtgcagcctctggaataactttcagtaactatgccatgtcttggattcgccagactccagagaagagactggagtgggtcgcaagtattagtagtggtggtagttatatctactattcagacagtgtgaagggtcgattcaccatttccagagacaatgccaggaacaccctgaacctgcaaatgagcagtctgaggtctgaggacacggccatgtattactgtgtaagaggggcgcatggaaattttgactactggggccaaggcaccactctcacagtctcctca

SEQ ID NO: 19 - protein, 136 aa, synthetic construct
  REGION 1..136: note = 14D12 heavy chain
MNFGLSLIFLVLILKGVQCEVMLVESGGALVKPGGSLKLSCAASGITFSNYAMSWIRQTPEKRLEWVASISSGGSYIYYSDSVKGRFTISRDNARNTLNLQMSSLRSEDTAMYYCVRGAHGNFDYWGQGTTLTVSS

SEQ ID NO: 20 - DNA, 393 bp, synthetic construct
  misc_feature 1..393: note = 14D12 light chain
  source 1..393: mol_type = other DNA
atggagtcagacacactcctgctatgggtgctgctgctctgggttccaggctccactggtgacattgtgctgacccaatctccagcttctttggctgtgtctctagggcagagagccaccatctcctgcagaaccagtgaaactgttgattatgatggcgatagttatatgaactggtaccaacagaaatcaggacagccacccaaactcctcatatctggtgcatccaacgtagagtctggggtccctgccaggttcagtggcagtgggtctgggacagacttcagcctcaacatccatcctgtggaggaggatgatattacaatgtatttctgtcagcaaaataggaagcttccgtatacgttcggatcggggaccaagctggaaatgaaa

SEQ ID NO: 21 - protein, 131 aa, synthetic construct
  REGION 1..131: note = 14D12 light chain
MESDTLLLWVLLLWVPGSTGDIVLTQSPASLAVSLGQRATISCRTSETVDYDGDSYMNWYQQKSGQPPKLLISGASNVESGVPARFSGSGSGTDFSLNIHPVEEDDITMYFCQQNRKLPYTFGSGTKLEMK

SEQ ID NO: 22 - protein, 117 aa, synthetic construct
  REGION 1..117: note = 14D12 heavy chain
EVMLVESGGALVKPGGSLKLSCAASGITFSNYAMSWIRQTPEKRLEWVASISSGGSYIYYSDSVKGRFTISRDNARNTLNLQMSSLRSEDTAMYYCVRGAHGNFDYWGQGTTLTVSS

SEQ ID NO: 23 - protein, 111 aa, synthetic construct
  REGION 1..111: note = 14D12 light chain
DIVLTQSPASLAVSLGQRATISCRTSETVDYDGDSYMNWYQQKSGQPPKLLISGASNVESGVPARFSGSGSGTDFSLNIHPVEEDDITMYFCQQNRKLPYTFGSGTKLEMK

SEQ ID NO: 24 - protein, 5 aa, synthetic construct
  REGION 1..5: note = 14D12 fragment
NYAMS

SEQ ID NO: 25 - protein, 17 aa, synthetic construct
  REGION 1..17: note = 14D12 fragment
SISSGGSYIYYSDSVKG

SEQ ID NO: 26 - protein, 8 aa, synthetic construct
  REGION 1..8: note = 14D12 fragment
GAHGNFDY

SEQ ID NO: 27 - protein, 15 aa, synthetic construct
  REGION 1..15: note = 14D12 fragment
RTSETVDYDGDSYMN

SEQ ID NO: 28 - protein, 7 aa, synthetic construct
  REGION 1..7: note = 14D12 fragment
GASNVES

SEQ ID NO: 29 - protein, 9 aa, synthetic construct
  REGION 1..9: note = 14D12 fragment
QQNRKLPYT

SEQ ID NO: 30 - DNA, 417 bp, synthetic construct
  misc_feature 1..417: note = 23F8 heavy chain
  source 1..417: mol_type = other DNA
atggactccaggctcaatttagttttccttgtcctttttttaaaaggtgtccagtgtgaggtgcagctggtggagtctgggggaggcttagtgaagcctggagggtcccggaaactctcctgtgcagcctctggattcactttcagtgactatggaatgcactgggttcgtcaggctccagagaagggactggagtgggttgcatacattagtagtggcagtagaaccatctactatgcagacacagtgaagggccgattcaccatctccagagacaatgccaagaacaccctgttcctgcaaatgaccagtctaaggtctgaggacacggccatgtattactgtgcaaggctgggctatggttacgactggtacttcgatgtctggggcacagggaccacggtcaccgtctcctca

SEQ ID NO: 31 - protein, 139 aa, synthetic construct
  REGION 1..139: note = 23F8 heavy chain
MDSRLNLVFLVLFLKGVQCEVQLVESGGGLVKPGGSRKLSCAASGFTFSDYGMHWVRQAPEKGLEWVAYISSGSRTIYYADTVKGRFTISRDNAKNTLFLQMTSLRSEDTAMYYCARLGYGYDWYFDVWGTGTTVTVSS

SEQ ID NO: 32 - DNA, 393 bp, synthetic construct
  misc_feature 1..393: note = 23F8 light chain
  source 1..393: mol_type = other DNA
atgagtcctgcccagttcctgtttctgttagtgctctggatgcgggaaaccaacggtgatgttgtgatgacccagactccactcactttgtcggttaccattggacaaccagcctccatctcctgcaagtcaagtcagagcctcttagatagtgatggaaggacatatttgaattggttgttacagaggccaggccagtctccaaagcgcctgatctatctggtgtctaaactggactctggagtccctgacaggttcactggcagtggatcagggacagatttcacactgaaaatcagcagagtggaggctgaggatttgggaatttattattgctggcaaggtacacattttcctcagacgttcggtggaggcaccaagctggaaatcaaa

SEQ ID NO: 33 - protein, 131 aa, synthetic construct
  REGION 1..131: note = 23F8 light chain
MSPAQFLFLLVLWMRETNGDVVMTQTPLTLSVTIGQPASISCKSSQSLLDSDGRTYLNWLLQRPGQSPKRLIYLVSKLDSGVPDRFTGSGSGTDFTLKISRVEAEDLGIYYCWQGTHFPQTFGGGTKLEIK

SEQ ID NO: 34 - protein, 120 aa, synthetic construct
  REGION 1..120: note = 23F8 heavy chain
EVQLVESGGGLVKPGGSRKLSCAASGFTFSDYGMHWVRQAPEKGLEWVAYISSGSRTIYYADTVKGRFTISRDNAKNTLFLQMTSLRSEDTAMYYCARLGYGYDWYFDVWGTGTTVTVSS

SEQ ID NO: 35 - protein, 112 aa, synthetic construct
  REGION 1..112: note = 23F8 light chain
DVVMTQTPLTLSVTIGQPASISCKSSQSLLDSDGRTYLNWLLQRPGQSPKRLIYLVSKLDSGVPDRFTGSGSGTDFTLKISRVEAEDLGIYYCWQGTHFPQTFGGGTKLEIK

SEQ ID NO: 36 - protein, 5 aa, synthetic construct
  REGION 1..5: note = 23F8 fragment
DYGMH

SEQ ID NO: 37 - protein, 17 aa, synthetic construct
  REGION 1..17: note = 23F8 fragment
YISSGSRTIYYADTVKG

SEQ ID NO: 38 - protein, 11 aa, synthetic construct
  REGION 1..11: note = 23F8 fragment
LGYGYDWYFDV

SEQ ID NO: 39 - protein, 16 aa, synthetic construct
  REGION 1..16: note = 23F8 fragment
KSSQSLLDSDGRTYLN

SEQ ID NO: 40 - protein, 7 aa, synthetic construct
  REGION 1..7: note = 23F8 fragment
LVSKLDS

SEQ ID NO: 41 - protein, 9 aa, synthetic construct
  REGION 1..9: note = 23F8 fragment
WQGTHFPQT

SEQ ID NO: 42 - DNA, 420 bp, synthetic construct
  misc_feature 1..420: note = 18B2 heavy chain
  source 1..420: mol_type = other DNA
atggaatggagcggggtctttatctttctcctgtcagtaactgcaggtgtccactcccaggtccagctgcagcagtctggagctgagctggtaaggcctgggacttcagtgaagatgtcctgcaaggctgctggatacaccttcactaactactggataggttgggtaaagcagaggcctggacatggccttgagtggattggagatatttaccctggaggtgtttatactaactacaatgagaacttcaagggcaaggccacactgacggcagacacatcctccagcacagcccacatgcagctcagcagcctgacatctgaggactctgccatctattactgtgtaagaggagggaagtatggtaactttttcgctatggactactggggtcaaggaacgtcagtcaccgtctcctca

SEQ ID NO: 43 - protein, 140 aa, synthetic construct
  REGION 1..140: note = 18B2 heavy chain
MEWSGVFIFLLSVTAGVHSQVQLQQSGAELVRPGTSVKMSCKAAGYTFTNYWIGWVKQRPGHGLEWIGDIYPGGVYTNYNENFKGKATLTADTSSSTAHMQLSSLTSEDSAIYYCVRGGKYGNFFAMDYWGQGTSVTVSS

SEQ ID NO: 44 - DNA, 396 bp, synthetic construct
  misc_feature 1..396: note = 18B2 light chain
  source 1..396: mol_type = other DNA
atgaggtgctctcttcagttcctgggggtgcttatgttctggatctctggagtcactggggatattgtgataacccaggatgaactctccaatcctgtcacttctggagaatcagtttccatctcctgcaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattactgtcaacaacttgtagagtatccattcacgttcggctcggggacaaaattggaaataaaa

SEQ ID NO: 45 - protein, 132 aa, synthetic construct
  REGION 1..132: note = 18B2 light chain
MRCSLQFLGVLMFWISGVTGDIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGSGTKLEIK

SEQ ID NO: 46 - protein, 121 aa, synthetic construct
  REGION 1..121: note = 18B2 heavy chain
QVQLQQSGAELVRPGTSVKMSCKAAGYTFTNYWIGWVKQRPGHGLEWIGDIYPGGVYTNYNENFKGKATLTADTSSSTAHMQLSSLTSEDSAIYYCVRGGKYGNFFAMDYWGQGTSVTVSS

SEQ ID NO: 47 - protein, 112 aa, synthetic construct
  REGION 1..112: note = 18B2 light chain
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGSGTKLEIK

SEQ ID NO: 48 - protein, 5 aa, synthetic construct
  REGION 1..5: note = 18B2 fragment
NYWIG

SEQ ID NO: 49 - protein, 17 aa, synthetic construct
  REGION 1..17: note = 18B2 fragment
DIYPGGVYTNYNENFKG

SEQ ID NO: 50 - protein, 12 aa, synthetic construct
  REGION 1..12: note = 18B2 fragment
GGKYGNFFAMDY

SEQ ID NO: 51 - protein, 16 aa, synthetic construct
  REGION 1..16: note = 18B2 fragment
RSSKSLLYKDGKTYLN

SEQ ID NO: 52 - protein, 7 aa, synthetic construct
  REGION 1..7: note = 18B2 fragment
LMSTRAS

SEQ ID NO: 53 - protein, 9 aa, synthetic construct
  REGION 1..9: note = 18B2 fragment
QQLVEYPFT

SEQ ID NO: 54 - DNA, 408 bp, synthetic construct
  misc_feature 1..408: note = 20F4 heavy chain
  source 1..408: mol_type = other DNA
atggcttgggtgtggaccttgctattcctgatggcagctgcccaaagtgcccaagcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccaccactgcctatttgcagatcaacaacctcaaaaatgaggacacggctacatatttctgtgcaagggcggctacggggtactttgactactggggccaaggcaccactctcacagtctcctca

SEQ ID NO: 55 - protein, 136 aa, synthetic construct
  REGION 1..136: note = 20F4 heavy chain
MAWVWTLLFLMAAAQSAQAQIQLVQSGPELKKPGETVKISCKASGYTFTNYGMNWVKQAPGKGLKWMGWINTYTGEPTYADDFKGRFAFSLETSATTAYLQINNLKNEDTATYFCARAATGYFDYWGQGTTLTVSS

SEQ ID NO: 56 - DNA, 396 bp, synthetic construct
  misc_feature 1..396: note = 20F4 light chain
  source 1..396: mol_type = other DNA
atggattcacaggcccaagttcttatgttgctgctgctatgggtatctggtacctgtggggacattgtgatgtcacagtctccatcctccctggctgtgtcagcaggagagaaggttactatgagctgcaagtccagtcagagccttttatatagtagcaatcaaaagaactacttggcctggtaccagcagaaaccagggcagtctcctaaactgttgatctactgggcatccactagggaatctggggtccctgaccgcttcacaggcagtggatcagggacagatttcactctcaccatcagcagtgtgaaggctgaagacctggccgtttattactgtcagcaatattataacgagtacacgttcggaggggggaccaagctggaaataaaa

SEQ ID NO: 57 - protein, 132 aa, synthetic construct
  REGION 1..132: note = 20F4 light chain
MDSQAQVLMLLLLWVSGTCGDIVMSQSPSSLAVSAGEKVTMSCKSSQSLLYSSNQKNYLAWYQQKPGQSPKLLIYWASTRESGVPDRFTGSGSGTDFTLTISSVKAEDLAVYYCQQYYNEYTFGGGTKLEIK

SEQ ID NO: 58 - protein, 117 aa, synthetic construct
  REGION 1..117: note = 20F4 heavy chain
QIQLVQSGPELKKPGETVKISCKASGYTFTNYGMNWVKQAPGKGLKWMGWINTYTGEPTYADDFKGRFAFSLETSATTAYLQINNLKNEDTATYFCARAATGYFDYWGQGTTLTVSS

SEQ ID NO: 59 - protein, 112 aa, synthetic construct
  REGION 1..112: note = 20F4 light chain
DIVMSQSPSSLAVSAGEKVTMSCKSSQSLLYSSNQKNYLAWYQQKPGQSPKLLIYWASTRESGVPDRFTGSGSGTDFTLTISSVKAEDLAVYYCQQYYNEYTFGGGTKLEIK

SEQ ID NO: 60 - protein, 5 aa, synthetic construct
  REGION 1..5: note = 20F4 fragment
NYGMN

SEQ ID NO: 61 - protein, 17 aa, synthetic construct
  REGION 1..17: note = 20F4 fragment
WINTYTGEPTYADDFKG

SEQ ID NO: 62 - protein, 8 aa, synthetic construct
  REGION 1..8: note = 20F4 fragment
AATGYFDY

SEQ ID NO: 63 - protein, 17 aa, synthetic construct
  REGION 1..17: note = 20F4 fragment
KSSQSLLYSSNQKNYLA

SEQ ID NO: 64 - protein, 7 aa, synthetic construct
  REGION 1..7: note = 20F4 fragment
WASTRES

SEQ ID NO: 65 - protein, 8 aa, synthetic construct
  REGION 1..8: note = 20F4 fragment
QQYYNEYT

SEQ ID NO: 66 - DNA, 438 bp, synthetic construct
  misc_feature 1..438: note = 14G11 heavy chain
  source 1..438: mol_type = other DNA
atgaagttgtggctgaactggattttccttgtaacacttttaaatggtatccagtgtgaggtgaagctggtggagtctggaggaggcttggtacagcctgggggttctctgagtctctcctgtgcagcttctggattcaccttcactgattactacatgagctgggtccgccagcctccagggaaggcacttgagtggttgggttttattagaaacaaagctaatggttacacaacagagtacagtgcatctgtgaagggtcggttcaccatctccagagataattcccaaagcatcctctatcttcaaatgaatgccctgagagctgaggacagtgccacttattactgtgcaagatataggcgggattactacggtagtcttaattactatactatggactactggggtcaaggaacctcagtcaccgtctcctca

SEQ ID NO: 67 - protein, 146 aa, synthetic construct
  REGION 1..146: note = 14G11 heavy chain
MKLWLNWIFLVTLLNGIQCEVKLVESGGGLVQPGGSLSLSCAASGFTFTDYYMSWVRQPPGKALEWLGFIRNKANGYTTEYSASVKGRFTISRDNSQSILYLQMNALRAEDSATYYCARYRRDYYGSLNYYTMDYWGQGTSVTVSS

SEQ ID NO: 68 - DNA, 381 bp, synthetic construct
  misc_feature 1..381: note = 14G11 light chain
  source 1..381: mol_type = other DNA
atgagtgtgcccactcaggtcctggggttgctgctgctgtggcttacaggtgccagatgtgacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaaaatcattatggtattcctctcacgttcggtgctgggaccaagctggagctgaaa

SEQ ID NO: 69 - protein, 127 aa, synthetic construct
  REGION 1..127: note = 14G11 light chain
MSVPTQVLGLLLLWLTGARCDIQMTQSPASLSASVGETVTITCRASENIYSYLAWYQQKQGKSPQLLVYNAKTLAEGVPSRFSGSGSGTQFSLKINSLQPEDFGSYYCQNHYGIPLTFGAGTKLELK

SEQ ID NO: 70 - protein, 127 aa, synthetic construct
  REGION 1..127: note = 14G11 heavy chain
EVKLVESGGGLVQPGGSLSLSCAASGFTFTDYYMSWVRQPPGKALEWLGFIRNKANGYTTEYSASVKGRFTISRDNSQSILYLQMNALRAEDSATYYCARYRRDYYGSLNYYTMDYWGQGTSVTVSS

SEQ ID NO: 71 - protein, 107 aa, synthetic construct
  REGION 1..107: note = 14G11 light chain
DIQMTQSPASLSASVGETVTITCRASENIYSYLAWYQQKQGKSPQLLVYNAKTLAEGVPSRFSGSGSGTQFSLKINSLQPEDFGSYYCQNHYGIPLTFGAGTKLELK

SEQ ID NO: 72 - protein, 5 aa, synthetic construct
  REGION 1..5: note = 14G11 fragment
DYYMS

SEQ ID NO: 73 - protein, 19 aa, synthetic construct
  REGION 1..19: note = 14G11 fragment
FIRNKANGYTTEYSASVKG

SEQ ID NO: 74 - protein, 16 aa, synthetic construct
  REGION 1..16: note = 14G11 fragment
YRRDYYGSLNYYTMDY

SEQ ID NO: 75 - protein, 11 aa, synthetic construct
  REGION 1..11: note = 14G11 fragment
RASENIYSYLA

SEQ ID NO: 76 - protein, 7 aa, synthetic construct
  REGION 1..7: note = 14G11 fragment
NAKTLAE

SEQ ID NO: 77 - protein, 9 aa, synthetic construct
  REGION 1..9: note = 14G11 fragment
QNHYGIPLT

SEQ ID NO: 78 - DNA, 411 bp, synthetic construct
  misc_feature 1..411: note = 13E11 heavy chain
  source 1..411: mol_type = other DNA
atggactccaggctcaatttagttttccttgtccttattttaaaaggtgtccagtgtgaggtgcagctggtggagtctgggggaggcttagtgaagcctggagggtcccggaaactctcctgtgcagcctctggattcactttcagtgactatggaatgcactgggttcgtcaggctccagagaaggggctggagtgggttgcatacattagtagtggcagtagtaccatctactatgcagacacagtgaagggccgattcaccatctccagagacaatgccaagaacaccctgttcctgcaaatgacgagtctaaggtctgaggacacggccatgtattactgtgcaaggccccgaagtgggaggtactttgactactggggccaaggcaccactctcacagtctcctca

SEQ ID NO: 79 - protein, 137 aa, synthetic construct
  REGION 1..137: note = 13E11 heavy chain
MDSRLNLVFLVLILKGVQCEVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARPRSGRYFDYWGQGTTLTVSS

SEQ ID NO: 80 - DNA, 393 bp, synthetic construct
  misc_feature 1..393: note = 13E11 light chain
  source 1..393: mol_type = other DNA
atgagtcctgcccagttcctgtttctgttagtgctctggattcaggaaaccaacggtgatgttatgatgacccagactccactcactttgtcggttaccattggacaaccagcttccatctcttgcaagtcaagtcagagcctcttagatagtaacggaaatacctatctgcattggttattacagaggccaggccagtctccaaagatcctaatctatctggtgtctaaactggactctggagtccctgacaggttcagtggcagtgggtcaggaacagatttcacactgaaaatcagcagagtggaggctgaggatttgggagtttattactgcttgcaaggtacacattttccgtacacgttcggaggggggaccaagctggaaataaaa

SEQ ID NO: 81 - protein, 131 aa, synthetic construct
  REGION 1..131: note = 13E11 light chain
MSPAQFLFLLVLWIQETNGDVMMTQTPLTLSVTIGQPASISCKSSQSLLDSNGNTYLHWLLQRPGQSPKILIYLVSKLDSGVPDRFSGSGSGTDFTLKISRVEAEDLGVYYCLQGTHFPYTFGGGTKLEIK

SEQ ID NO: 82 - protein, 118 aa, synthetic construct
  REGION 1..118: note = 13E11 heavy chain
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARPRSGRYFDYWGQGTTLTVSS

SEQ ID NO: 83 - protein, 112 aa, synthetic construct
  REGION 1..112: note = 13E11 light chain
DVMMTQTPLTLSVTIGQPASISCKSSQSLLDSNGNTYLHWLLQRPGQSPKILIYLVSKLDSGVPDRFSGSGSGTDFTLKISRVEAEDLGVYYCLQGTHFPYTFGGGTKLEIK

SEQ ID NO: 84 - protein, 5 aa, synthetic construct
  REGION 1..5: note = 13E11 fragment
DYGMH

SEQ ID NO: 85 - protein, 17 aa, synthetic construct
  REGION 1..17: note = 13E11 fragment
YISSGSSTIYYADTVKG

SEQ ID NO: 86 - protein, 9 aa, synthetic construct
  REGION 1..9: note = 13E11 fragment
PRSGRYFDY

SEQ ID NO: 87 - protein, 16 aa, synthetic construct
  REGION 1..16: note = 13E11 fragment
KSSQSLLDSNGNTYLH

SEQ ID NO: 88 - protein, 7 aa, synthetic construct
  REGION 1..7: note = 13E11 fragment
LVSKLDS

SEQ ID NO: 89 - protein, 9 aa, synthetic construct
  REGION 1..9: note = 13E11 fragment
LQGTHFPYT

SEQ ID NO: 90 - DNA, 393 bp, synthetic construct
  misc_feature 1..393: note = 10B1 heavy chain
  source 1..393: mol_type = other DNA
atgaaatgcagctgggttatcttcttcctgatggcagtggttacaggggtcaattcagaggttcagctgcagcagtctggggcagagcttgtgaagccaggggcctcagtcaagttgtcctgcacagcttctggcttcaacattaaagacacctatatacactgggtgaaacagaggcctgaacagggcctggagtggattggaaggattgatcctgcgaatggtaatactatatatgcctcaaagttccagggcaaggcccctataacagcagtcacatcatccaacacagcctacatgcagttcagcagcctgacatctggggacactgccgtctattactgtactgctatggactactggggtcaaggaacctcagtcaccgtctcctca

SEQ ID NO: 91 - protein, 131 aa, synthetic construct
  REGION 1..131: note = 10B1 heavy chain
MKCSWVIFFLMAVVTGVNSEVQLQQSGAELVKPGASVKLSCTASGFNIKDTYIHWVKQRPEQGLEWIGRIDPANGNTIYASKFQGKAPITAVTSSNTAYMQFSSLTSGDTAVYYCTAMDYWGQGTSVTVSS

SEQ ID NO: 92 - DNA, 393 bp, synthetic construct
  misc_feature 1..393: note = 10B1 light chain
  source 1..393: mol_type = other DNA
atgagtcctgcccagttcctgtttctgctagtgctcgcgattcaggaaaccaacggtgatgttgtgatgactcagaccccactcactttgtcggttaccattggacaaccagcctccatctcttgcaaatcaagtcagagcctcttacatagtaatggaaagacatatttgaattggttattacagaggccaggccagtctccaaagctcctaatctatctggtgtctaaactggattctggagtccctgacaggttcagtggcagtggatcagggacagatttcacactgaaaatcagcagagtggaggctgaggatttgggagtttattactgcttgcaagctacacattttcctcatacgttcggatcggggaccaagctggaaataaaa

SEQ ID NO: 93 - protein, 131 aa, synthetic construct
  REGION 1..131: note = 10B1 light chain
MSPAQFLFLLVLAIQETNGDVVMTQTPLTLSVTIGQPASISCKSSQSLLHSNGKTYLNWLLQRPGQSPKLLIYLVSKLDSGVPDRFSGSGSGTDFTLKISRVEAEDLGVYYCLQATHFPHTFGSGTKLEIK

SEQ ID NO: 94 - protein, 112 aa, synthetic construct
  REGION 1..112: note = 10B1 heavy chain
EVQLQQSGAELVKPGASVKLSCTASGFNIKDTYIHWVKQRPEQGLEWIGRIDPANGNTIYASKFQGKAPITAVTSSNTAYMQFSSLTSGDTAVYYCTAMDYWGQGTSVTVSS

SEQ ID NO: 95 - protein, 112 aa, synthetic construct
  REGION 1..112: note = 10B1 light chain
DVVMTQTPLTLSVTIGQPASISCKSSQSLLHSNGKTYLNWLLQRPGQSPKLLIYLVSKLDSGVPDRFSGSGSGTDFTLKISRVEAEDLGVYYCLQATHFPHTFGSGTKLEIK

SEQ ID NO: 96 - protein, 5 aa, synthetic construct
  REGION 1..5: note = 10B1 fragment
DTYIH

SEQ ID NO: 97 - protein, 17 aa, synthetic construct
  REGION 1..17: note = 10B1 fragment
RIDPANGNTIYASKFQG

SEQ ID NO: 98 - skipped

SEQ ID NO: 99 - protein, 16 aa, synthetic construct
  REGION 1..16: note = 10B1 fragment
KSSQSLLHSNGKTYLN

SEQ ID NO: 100 - protein, 7 aa, synthetic construct
  REGION 1..7: note = 10B1 fragment
LVSKLDS

SEQ ID NO: 101 - protein, 9 aa, synthetic construct
  REGION 1..9: note = 10B1 fragment
LQATHFPHT

SEQ ID NO: 102 - DNA, 393 bp, synthetic construct
  misc_feature 1..393: note = 7E11 heavy chain
  source 1..393: mol_type = other DNA
atgaaatgcagctgggttatcttcttcctgatggcagtggttacaggggtcaattcagaggttcagctgcagcagtctggggcagagcttgtgaagccaggggcctcagtcaagttgtcctgcacagcttctggcttcaacattaaagacacctatatacactgggtgaagcagaggcctgaacagggcctggagtggattggaaggattgatcctgcgaatggtaatattatatatgcctcaaagttccagggcgaggccactataacagcagacacatcatccaacacagcctacatgcagctcagcagcctgacatctggggacactgccgtctattactgtagcgctatggactactggggtcaaggaacctcagtcaccgtctcctca

SEQ ID NO: 103 - protein, 131 aa, synthetic construct
  REGION 1..131: note = 7E11 heavy chain
MKCSWVIFFLMAVVTGVNSEVQLQQSGAELVKPGASVKLSCTASGFNIKDTYIHWVKQRPEQGLEWIGRIDPANGNIIYASKFQGEATITADTSSNTAYMQLSSLTSGDTAVYYCSAMDYWGQGTSVTVSS

SEQ ID NO: 104 - DNA, 393 bp, synthetic construct
  misc_feature 1..393: note = 7E11 light chain
  source 1..393: mol_type = other DNA
atgagtcctgcccagttcctgtttctgctagtgctctcgattcaggaaaccaacggtgatgttgtgatgactcagaccccactcactttgtcgcttaccattggacaaccagcctccatctcttgcaaatcaagtcagagcctcttacatagtaatggaaagacatatttgaattggttattacagaggccaggccagtctcctaagctcctcatctatctggtgtctaaactggattctggagtccctgacaggttcagtggcagtggatcagggacagatttcacactgaaaatcagcagagtggaggctgaggatttgggagtttattactgcttgcaagctacacattttcctcatacgttcggatcggggaccaagctggaaataaaa

SEQ ID NO: 105 - protein, 131 aa, synthetic construct
  REGION 1..131: note = 7E11 light chain
MSPAQFLFLLVLSIQETNGDVVMTQTPLTLSLTIGQPASISCKSSQSLLHSNGKTYLNWLLQRPGQSPKLLIYLVSKLDSGVPDRFSGSGSGTDFTLKISRVEAEDLGVYYCLQATHFPHTFGSGTKLEIK

SEQ ID NO: 106 - protein, 112 aa, synthetic construct
  REGION 1..112: note = 7E11 heavy chain
EVQLQQSGAELVKPGASVKLSCTASGFNIKDTYIHWVKQRPEQGLEWIGRIDPANGNIIYASKFQGEATITADTSSNTAYMQLSSLTSGDTAVYYCSAMDYWGQGTSVTVSS

SEQ ID NO: 107 - protein, 112 aa, synthetic construct
  REGION 1..112: note = 7E11 light chain
DVVMTQTPLTLSLTIGQPASISCKSSQSLLHSNGKTYLNWLLQRPGQSPKLLIYLVSKLDSGVPDRFSGSGSGTDFTLKISRVEAEDLGVYYCLQATHFPHTFGSGTKLEIK

SEQ ID NO: 108 - protein, 5 aa, synthetic construct
  REGION 1..5: note = 7E11 fragment
DTYIH

SEQ ID NO: 109 - protein, 17 aa, synthetic construct
  REGION 1..17: note = 7E11 fragment
RIDPANGNIIYASKFQG

SEQ ID NO: 110 - skipped

SEQ ID NO: 111 - protein, 16 aa, synthetic construct
  REGION 1..16: note = 7E11 fragment
KSSQSLLHSNGKTYLN

SEQ ID NO: 112 - protein, 7 aa, synthetic construct
  REGION 1..7: note = 7E11 fragment
LVSKLDS

SEQ ID NO: 113 - protein, 9 aa, synthetic construct
  REGION 1..9: note = 7E11 fragment
LQATHFPHT

SEQ ID NO: 114 - DNA, 414 bp, synthetic construct
  misc_feature 1..414: note = 3E6 heavy chain
  source 1..414: mol_type = other DNA
atgaagttgtggctgaactggattttccttgtaacacttttaaatggtatccagtgtgaggtgaagctggtggagtctggaggaggcttggtacagcctgggggttctctgagactctcctgtgcaacttctgggttcaccttcactgattactacatgagctgggtccgccagcctccaggaaaggcacttgagtggatgggttttattagaaacaaagctaaaggttacacaacagattacagtgcgtctgtgaagggtcggttcaccatctccagagatgattcccaaagcatcctctatcttcaaatgaacaccctgagacctgaggacagtgccacttattactgtgcaagaaactatgactattctatggactactggggtcaaggaacctcagtcaccgtctcctca

SEQ ID NO: 115 - protein, 138 aa, synthetic construct
  REGION 1..138: note = 3E6 heavy chain
MKLWLNWIFLVTLLNGIQCEVKLVESGGGLVQPGGSLRLSCATSGFTFTDYYMSWVRQPPGKALEWMGFIRNKAKGYTTDYSASVKGRFTISRDDSQSILYLQMNTLRPEDSATYYCARNYDYSMDYWGQGTSVTVSS

SEQ ID NO: 116 - DNA, 381 bp, synthetic construct
  misc_feature 1..381: note = 3E6 light chain
  source 1..381: mol_type = other DNA
atgagtgtgctcactcaggtcctggcgttgctgctgctgtggcttacaggtgccagatgtgacatccagctgactcagtctccagcctccctatctgcatctgtgggagaaactgtcaccatcacttgtcgagcaagtgataatattcacaaatatttagcatggtatcagcagaaacagggaaagtctcctcagcgcctggtctataatgcaaaaaccttagcagatggtgtgccatcaaggttcaatggcagtggatcaggaacacaatattctctcaagatcaatagcctgcagcctgaagattttgggatttattactgtcaacatttttggagtactccgctcacgttcggtgctgggaccaagctggagctgaaa

SEQ ID NO: 117 - protein, 127 aa, synthetic construct
  REGION 1..127: note = 3E6 light chain
MSVLTQVLALLLLWLTGARCDIQLTQSPASLSASVGETVTITCRASDNIHKYLAWYQQKQGKSPQRLVYNAKTLADGVPSRFNGSGSGTQYSLKINSLQPEDFGIYYCQHFWSTPLTFGAGTKLELK

SEQ ID NO: 118 - protein, 119 aa, synthetic construct
  REGION 1..119: note = 3E6 heavy chain
EVKLVESGGGLVQPGGSLRLSCATSGFTFTDYYMSWVRQPPGKALEWMGFIRNKAKGYTTDYSASVKGRFTISRDDSQSILYLQMNTLRPEDSATYYCARNYDYSMDYWGQGTSVTVSS

SEQ ID NO: 119 - protein, 107 aa, synthetic construct
  REGION 1..107: note = 3E6 light chain
DIQLTQSPASLSASVGETVTITCRASDNIHKYLAWYQQKQGKSPQRLVYNAKTLADGVPSRFNGSGSGTQYSLKINSLQPEDFGIYYCQHFWSTPLTFGAGTKLELK

SEQ ID NO: 120 - protein, 5 aa, synthetic construct
  REGION 1..5: note = 3E6 fragment
DYYMS

SEQ ID NO: 121 - protein, 19 aa, synthetic construct
  REGION 1..19: note = 3E6 fragment
FIRNKAKGYTTDYSASVKG

SEQ ID NO: 122 - protein, 8 aa, synthetic construct
  REGION 1..8: note = 3E6 fragment
NYDYSMDY

SEQ ID NO: 123 - protein, 11 aa, synthetic construct
  REGION 1..11: note = 3E6 fragment
RASDNIHKYLA

SEQ ID NO: 124 - protein, 7 aa, synthetic construct
  REGION 1..7: note = 3E6 fragment
NAKTLAD

SEQ ID NO: 125 - protein, 9 aa, synthetic construct
  REGION 1..9: note = 3E6 fragment
QHFWSTPLT

(19) United States
(12) Patent Application Publication
(10) Pub. No.: US 2024/0092877 A1
(43) Pub. Date: Mar. 21, 2024

(54) METHODS AND COMPOSITIONS FOR PERTUSSIS DIAGNOSIS

(71) Applicant: DX DISCOVERY INC., Reno, NV (US)

(72) Inventor: Amanda Burnham-Marusich, Reno, NV (US)

(21) Appl. No.: 18/510,115

(22) Filed: Nov. 15, 2023

Related U.S. Application Data
(63) Continuation of application No. 17/058,446, filed on Nov. 24, 2020, now Pat. No. 11,851,479.
(60) Provisional application No. 62/686,412, filed on Jun. 18, 2018.
(60) Provisional application No. 62/829,802, filed on Apr. 5, 2019.

(51) Int. Cl. C07K 16/12; G01N 33/543; G01N 33/569

(52) U.S. Cl. CPC C07K 16/1225; G01N 33/54388; G01N 33/56911; A61K 2039/505

(57) ABSTRACT

Compositions and methods for the detection of Bordetella pertussis and diagnosing pertussis are disclosed.

This application is a continuation application of U.S. patent application Ser. No. 17/058,446, filed Nov. 24, 2020, which is a § 371 application of PCT/US2019/037618, filed Jun. 18, 2019, which claims priority under 35 U.S.C. § 119(e) to U.S. Provisional Patent Application No. 62/686,412, filed on Jun. 18, 2018, and U.S. Provisional Patent Application No. 62/829,802, filed on Apr. 5, 2019. The foregoing applications are incorporated by reference herein.

Incorporated herein by reference in its entirety is the Sequence Listing being concurrently submitted as a XML file named SeqList, created Nov. 14, 2023, and having a size of 165,820 bytes.

This invention was made with government support under grant numbers

R43AI109891 and R44AI109891 awarded by the National Institutes of Health. The government has certain rights in the invention.

### FIELD OF THE INVENTION

This invention relates generally to the field of pertussis. Specifically, the invention provides novel compositions and methods for the early diagnosis of pertussis.

### BACKGROUND OF THE INVENTION

Pertussis is a respiratory disease caused by the gram-negative bacterium Bordetella pertussis. It is airborne, highly contagious, and responsible for an annual 18.4 million illnesses and 254,000 deaths worldwide (Warfel, et al. (2012) J. Infect. Dis., 206(6):902-6; Mertsola, et al. (1983) J. Pediatr., 103(3):359-63; Wirsing von Konig, et al. (1998) Eur. J. Pediatr., 157(5):391-4). Globally, pertussis is one of the leading causes of death for children under 5 years old (Black, et al. (2010) Lancet 375(9730):1969-87).

Pertussis incidence in the United States has been increasing since the early 1980s (Black, S. (1997) Pediatr. Infect. Dis. J., 16(4 Suppl):585-9; Crowcroft, et al. (2006) Lancet 367(9526):1926-36). Despite high vaccine coverage, there were still over 48,000 cases reported in the U.S. in 2012, which is the highest number since 1955 (Centers for Disease Control and Prevention (2013) MMWR Morb. Mortal Wkly. Rep., 62(33):669-82; Centers for Disease Control and Prevention (1980) MMWR Morb. Mortal Wkly. Rep., 28(54)). Moreover, reported cases represent a large underestimate of pertussis infections (Cherry, et al. (2005) Pediatr. Infect. Dis. J., 24(5 Suppl):S25-34; van den Brink, et al. (2014) BMC Infect. Dis.,14:526). Unfortunately neither vaccination nor previous infection provide life-long immunity to pertussis (Wendelboe, et al. (2005) Pediatr. Infect. Dis. J., 24(5 Suppl):S58-61). In particular, vaccine-induced immunity wanes after 4-12 years (Wendelboe, et al. (2005) Pediatr. Infect. Dis. J., 24(5 Suppl):S58-61), leaving many children and adults vulnerable to infection as well as household infants who are too young to have yet received the vaccine.

One of the largest obstacles to reducing the burden of pertussis is early diagnosis (Crowcroft, et al. (2006) Lancet 367(9526):1926-36; Cherry, et al. (2005) Pediatr. Infect. Dis. J., 24(5 Suppl):S25-34; Tondella, et al. (2009) Vaccine 27(6):803-14; Centers for Disease Control and Prevention (1997) MMWR Morb. Mortal Wkly. Rep., 46(35):822-6; Forsyth, et al. (2007) Vaccine 25(14):2634-42). Patient treatment and outbreak containment are effective, but only if initiated early (Tiwari, et al. (2005) MMWR Recomm. Rep., 54(RR-14):1-16; von Konig, C. H. (2005) Pediatr. Infect. Dis. J., 24(5 Suppl):S66-8).

However, prompt diagnosis of early pertussis is challenging because its symptoms are non-specific and because there are no assays that can rapidly diagnose pertussis at the point-of-care (POC). For example, bacterial culture, while being suitable for early diagnosis, is a very slow assay that requires 5-7 days at a site not at the point of care. Serological tests, while sensitive, cannot be used to detect early disease because patient antibodies are required. Various PCR or other DNA amplification based assays such as RT-PCR, helicase-dependent amplification (HDA) (e.g., AmpliVue® Bordetella Assay (Quidel, San Diego, CA)), nested multiplex PCR (e.g., FilmArray® Respiratory Panel (BioFire, Salt Lake City, Utah)), and loop mediated isothermal amplification (LAMP) (e.g., illumigene® Pertussis DNA Amplification Assay (Meridian Bioscience, London, England)) are available, but have many drawbacks. Indeed, these assays: 1) can be very expensive; 2) are not point of care assays as they are generally performed in a hospital or off-site lab; and 3) do not report on the antibiotic susceptibility of the B. pertussis. In view of the foregoing, it is clear that improved methods for early diagnosis of pertussis are needed.

### SUMMARY OF THE INVENTION

In accordance with one aspect of the instant invention, antibodies or antigen binding fragment thereof specific for tracheal colonization factor A (TcfA) are provided. In a particular embodiment, the anti-TcfA antibody or fragment thereof specifically binds amino acids 140-160, amino acids 229-240, amino acids 288-304, amino acids 286-321, amino acids 289-324, amino acids 305-323, amino acids 322-330, or amino acids 337-345 of TcfA. In a particular embodiment, the anti-TcfA antibody or fragment thereof specifically binds amino acids 140-150, amino acids 148-159, amino acids 151-156, amino acids 151-159, amino acids 229-240, amino acids 289-300, amino acids 305-312, amino acids 286-321, amino acids 289-324, amino acids 289-294, amino acids 292-300, amino acids 307-315, amino acids 310-315, amino acids 313-321, amino acids 322-330, or amino acids 337-345 of TcfA. The anti-TcfA antibodies of the instant invention may be conjugated to a detectable label such as a gold nanoparticle. Composition comprising an anti-TcfA antibody of the instant invention and a carrier are also provided.

In accordance with another aspect of the instant invention, methods of detecting Bordetella pertussis in a sample are provided. The methods comprise contacting the sample with an anti-TcfA antibody. Generally, the sample is a biological sample obtained from a subject. In a particular embodiment, the biological sample is a nasopharyngeal swab, aspirate, or wash.

In accordance with another aspect of the instant invention, methods for inhibiting, treating, and/or preventing pertussis and/or a B. pertussis infection in a subject in need thereof are provided. The methods comprise administering an anti-TcfA antibody of the instant invention to the subject. The method may further comprise administering antibiotics to the subject.

In accordance with yet another aspect of the instant invention, immunoassays for detecting B. pertussis are provided. The immunoassays comprise at least one anti-TcfA antibody of the instant invention. In a particular embodiment, the immunoassay is a lateral flow immunoassay test strip. In a particular embodiment, the immunoassay comprises a conjugated antibody which specifically binds amino acids 139-150 or amino acids 151-156 of TcfA. In a particular embodiment, the immunoassay comprises a test line antibody which specifically binds amino acids 289-324 of TcfA, amino acids 289-294 of TcfA, amino acids 292-300 of TcfA, and/or amino acids 322-330 of TcfA. In a particular embodiment, the immunoassay comprises a test line antibody which specifically binds amino acids 289-324, 229-240, 289-300, and/or 304-312 of TcfA (e.g., the same epitope as 13E11) and a test line antibody which specifically binds amino acids 288-304 or 292-300 (e.g., the same epitope as 14D12), particularly with a conjugated antibody which specifically binds amino acids 140-160 or 139-150 of TcfA (e.g., the same epitope as 10B1). In accordance with another aspect of the instant invention, methods of detecting Bordetella pertussis in a sample using the immunoassays are provided.

### BRIEF DESCRIPTION OF THE DRAWINGS

FIG. 1 provides a graph of an enzyme-linked immunosorbent assay (ELISA) using anti-tracheal colonization factor A (TcfA) antibodies against lysates of Bordetella pertussis, Moraxella catarrhalis, Streptococcus pneumoniae, Streptococcus pyogenes, Staphylococcus aureus, Haemophilus influenza, Escherichia coli, or a negative control (lysis buffer) at the indicated dilutions.

FIG. 2 provides images of a Western blot analysis using anti-TcfA antibodies on B. pertussis conditioned media (CM) and B. pertussis lysates. * indicates secreted TcfA isoform and arrowhead indicates cell-associated TcfA isoform.

FIG. 3A provides an image of a lateral flow immunoassay (LFI) using polyclonal anti-TcfA antibodies. The amount of B. pertussis colony forming units (CFUs) is indicated. FIG. 3B provides a graph of a quantitative limit of detection analysis of the lateral flow immunoassay (LFI) using anti-TcfA antibodies. The amount of B. pertussis colony forming units (CFUs) is indicated versus the band intensity.

FIG. 4 shows the reactivity of purified anti-TcfA monoclonal antibodies (mAbs) by ELISA with immobilized antigen. The immobilized antigens were formaldehyde-inactivated B. pertussis cells (Tohama I strain), recombinant TcfA containing a histidine tag, TcfA peptides (amino acids 140-160, 288-304, or 305-323) conjugated to bovine serum albumin (BSA), or control BSA. Data is the average of three independent experiments. ¹SS CM=1:128 dilution in phosphate buffered saline (PBS) pH 7.4 of clarified, 0.2 μm-filtered supernatant of B. pertussis (165 strain) Stainer-Scholte liquid cultures. ²SS Media=1:128 dilution in PBS pH 7.4 of Stainer-Scholte uninocculated medium. Shading from dark to light indicates high to low signal.

FIG. 5 shows the reactivity of anti-TcfA mAb based LFIs with formaldehyde-inactivated B. pertussis cells in phosphate buffered saline (PBS) at an OD₆₀₀ of 2.0. All LFIs that produced more signal with B. pertussis cells in PBS than with PBS alone (as determined by visual inspection) were quantified with a Qiagen ESEQuant Lateral Flow Reader running the Lateral Flow Studio Software suite. All LFI tests were performed a second time on a second day. Grey boxes indicate successful LFIs, as defined by meeting both of the following metrics: 1) had quantified signal with B. pertussis cells that was greater than twice the signal seen with PBS alone (signal comparisons were done with the averages of the two replicates tested), and 2) had quantified signal with PBS of less than 100 units (average of two replicates). White boxes indicate LFIs whose signal did not meet both of the former metrics.

FIG. 6 provides the amino acid sequence (SEQ ID NO: 1) of TcfA from B. pertussis. Underlined sequences are antibody epitopes.

FIG. 7 provides a graph of the analytical sensitivity of the mAB-based LFI for viable B. pertussis (Tohama I) in extraction buffer using monoclonal Abs. The detection limit of the LFI was determined to be 3×10⁵ CFU/mL (1.8×10⁴ CFU per LFI test). Viable B. pertussis (Tohama I strain) cells were suspended in phosphate buffered saline (PBS) at 11 different OD₆₀₀ concentrations. Cell suspensions were then mixed with extraction buffer for 5 minutes and then analyzed by LFI. Each concentration was evaluated on 20 replicate LFIs. All LFIs were from one lot. Each LFI was interpreted visually by three readers who were blind as to the concentration being tested. LFIs interpreted as positive by 3 of 3 blind readers were categorized as “Positive”. Nonlinear regression analysis using a 4-parameter logistic model was used to determine the line of best fit, and the equation for the line was used to calculate the concentration at which 95% of the LFIs would be reported as positive (C95; detection limit).

FIG. 8 provides an image of a LFI using monoclonal anti-TcfA antibodies. Lanes 1 and 2: formaldehyde-inactivated B. pertussis cells (Tohama I strain) cells in PBS (OD₆₀₀ of 0.1) vs. PBS alone. Lanes 3 and 4: clarified, 0.2 μm-filtered supernatant from B. pertussis (strain 165) cultures in Stainer-Scholte medium vs. uninoculated Stainer-Scholte medium (both diluted 1:128 in PBS). Lanes 5 and 6: formaldehyde-inactivated B. pertussis cells (Tohama I strain) in PBS (OD₆₀₀ of 0.1) lysed for 5 minutes in extraction buffer vs. extraction buffer alone.

FIG. 9 provides an image of a LFI using monoclonal anti-TcfA antibodies. Nasopharyngeal washes from baboons challenged with B. pertussis and containing the listed CFU per LFI testing volume were incubated with extraction buffer for 5 minutes at room temperature. LFI development time was 15 minutes.

FIG. 10 provides a graph showing the sensitivity of the mAb-based LFI with challenged baboon nasopharyngeal washes containing the indicated CFU. All infected baboon nasopharyngeal washes with ≥5×10⁵ CFU/mL were positive by LFI (top horizontal reference line), and all infected baboon nasopharyngeal washes with ≤3.5×10⁴ CFU/mL were negative by LFI (bottom horizontal reference line). A total of 30 nasopharyngeal washes from baboons challenged with B. pertussis were analyzed by LFI in duplicate. The LFI produced no false-positives with 11 baboon nasopharyngeal washes (tested in on duplicate LFIs) that had 0 CFU/mL (i.e. the LFI had 100% specificity).

FIGS. 11A-11I provide amino acid and nucleotide sequences of anti-TcfA antibodies. CDRs were determined by Kabat. Framework regions are underlined. FIG. 11A provides the nucleotide (SEQ ID NO: 18) and amino acid (SEQ ID NO: 19) sequences of the heavy chain and the nucleotide (SEQ ID NO: 20) and amino acid (SEQ ID NO: 21) sequences of the light chain of the 14D12 antibody. FIG. 11B provides the nucleotide (SEQ ID NO: 30) and amino acid (SEQ ID NO: 31) sequences of the heavy chain and the nucleotide (SEQ ID NO: 32) and amino acid (SEQ ID NO: 33) sequences of the light chain of the 23F8 antibody. FIG. 11C provides the nucleotide (SEQ ID NO: 42) and amino acid (SEQ ID NO: 43) sequences of the heavy chain and the nucleotide (SEQ ID NO: 44) and amino acid (SEQ ID NO: 45) sequences of the light chain of the 18B2 antibody. FIG. 11D provides the nucleotide (SEQ ID NO: 54) and amino acid (SEQ ID NO: 55) sequences of the heavy chain and the nucleotide (SEQ ID NO: 56) and amino acid (SEQ ID NO: 57) sequences of the light chain of the 20F4 antibody. FIG. 11E provides the nucleotide (SEQ ID NO: 66) and amino acid (SEQ ID NO: 67) sequences of the heavy chain and the nucleotide (SEQ ID NO: 68) and amino acid (SEQ ID NO: 69) sequences of the light chain of the 14G11 antibody. FIG. 11F provides the nucleotide (SEQ ID NO: 78) and amino acid (SEQ ID NO: 79) sequences of the heavy chain and the nucleotide (SEQ ID NO: 80) and amino acid (SEQ ID NO: 81) sequences of the light chain of the 13E11 antibody. FIG. 11G provides the nucleotide (SEQ ID NO: 90) and amino acid (SEQ ID NO: 91) sequences of the heavy chain and the nucleotide (SEQ ID NO: 92) and amino acid (SEQ ID NO: 93) sequences of the light chain of the 10B1 antibody. FIG. 11H provides the nucleotide (SEQ ID NO: 102) and amino acid (SEQ ID NO: 103) sequences of the heavy chain and the nucleotide (SEQ ID NO: 104) and amino acid (SEQ ID NO: 105) sequences of the light chain of the 7E11 antibody. FIG. 11I provides the nucleotide (SEQ ID NO: 114) and amino acid (SEQ ID NO: 115) sequences of the heavy chain and the nucleotide (SEQ ID NO: 116) and amino acid (SEQ ID NO: 117) sequences of the light chain of the 3E6 antibody.

### DETAILED DESCRIPTION OF THE INVENTION

Herein, a point-of-care, lateral flow immunoassay (LFI) diagnostic for early pertussis that enables immediate treatment initiation (e.g., during the patient's initial clinic visit) is provided. This point-of-care assay for detection of early pertussis will improve patient care and public health. It is well established that disease severity and duration can be reduced with pertussis if patients receive antibiotic treatment early (Tiwari, et al. (2005) MMWR Recomm. Rep., 54(RR-14):1-16; von Konig, C. H. (2005) Pediatr. Infect. Dis. J., 24(5 Suppl):566-8; Mattoo, et al. (2005) Clin. Microbiol. Rev., 18(2):326-82; Hewlet, et al. (2005) N. Engl. J. Med., 352(12):1215-22). For infants, early diagnosis would also save lives. Currently, infants require more doctor visits to reach a pertussis diagnosis than do older patients (Lee, et al. (2000) Arch. Fam. Med., 9(10):989-96). Infants also have the highest risk of mortality and severe neurological complications (Tanaka, et al. (2003) JAMA 290(22):2968-75). For infants, early diagnosis would enable not only earlier treatment with antibiotics, but also key supportive care for dehydration and malnutrition (Crowcroft, et al. (2006) Lancet 367(9526):1926-36; Hewlet, et al. (2005) N. Engl. J. Med., 352(12):1215-22).

Early diagnosis can change the course of an outbreak because patients are most infectious from the start of nonspecific symptoms until three weeks after paroxysmal cough onset (Tiwari, et al. (2005) MMWR Recomm. Rep., 54(RR-14):1-16). Antibiotic treatment eliminates culturable bacteria from the nasopharynx (Bergquist, et al. (1987) Pediatr. Infect. Dis. J., 6(5):458-61), which decreases the patients' infectious period and limits transmission (Wirsing von Konig, et al. (1998) Eur. J. Pediatr., 157(5):391-4). Moreover, once patients are diagnosed, their close contacts can receive prophylactic antibiotics (Tiwari, et al. (2005) MMWR Recomm. Rep., 54(RR-14):1-16; von Konig, C. H. (2005) Pediatr. Infect. Dis. J., 24(5 Suppl):S66-8). Thus, early diagnosis would reduce the size of pertussis outbreaks.

Minimizing outbreak size through early diagnosis would also reduce the economic burden of pertussis. More than $17 billion were spent on pertussis costs (direct and indirect) from 2001-2010 in the U.S. (Purdy, et al. (2004) Clin. Infect. Dis., 39(1):20-8). Preventing infant cases is particularly important because 70% of infants with pertussis become hospitalized (Tanaka, et al. (2003) JAMA 290(22):2968-75) and each infant hospital stay costs an average of $10,000, excluding outpatient direct and societal indirect costs (O'Brien, et al. (2005) BMC Infect. Dis., 5:57).

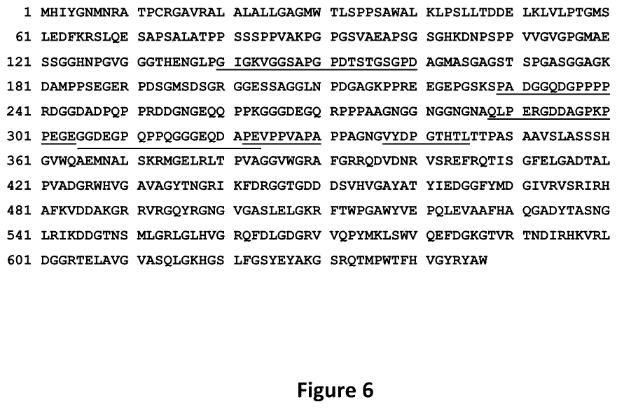

In accordance with one aspect of the instant invention, anti-tracheal colonization factor A (TcfA) antibodies and fragments thereof are provided. The anti-TcfA antibodies may be monoclonal or polyclonal. In a particular embodiment, the antibody or fragment thereof is immunologically specific for TcfA of Bordetella pertussis (e.g., Tohama I strain). Amino acid and nucleotide sequences of TcfA are provided in GenBank Accession No. NP 879974 and Gene ID: 2666888. FIG. 6 provides an amino acid sequence for TcfA (SEQ ID NO: 1) and certain anti-TcfA antibody epitopes. The anti-TcfA antibodies or fragments thereof may recognize a linear epitope or a conformational epitope, particularly a linear epitope. In a particular embodiment, the anti-TcfA antibody or fragment thereof recognizes a linear epitope. In a particular embodiment, the anti-TcfA antibody or fragment thereof is immunologically specific for a polypeptide comprising amino acids 140-160, amino acids 288-304, amino acids 305-323, amino acids 322-330, or amino acids 337-345 of TcfA. In a particular embodiment, the anti-TcfA antibody or fragment thereof is immunologically specific for a polypeptide comprising amino acids 139-150, amino acids 148-159, amino acids 151-156, amino acids 151-159, amino acids 229-240, amino acids 289-300, amino acids 304-312, amino acids 286-321, amino acids 289-324, amino acids 289-294, amino acids 292-300, amino acids 307-315, amino acids 310-315, amino acids 313-321, amino acids 322-330, or amino acids 337-345 of TcfA. The above epitopes may be longer or shorter than the above identified sequences by 0, 1, 2, 3, 4, 5, 6, 7, 8, 9, 10, or more amino acids, particularly 1, 2, 3, 4, or 5 amino acids, at the N-terminus and/or C-terminus of the sequence. In another embodiment, the above epitopes have at least 90%, 95%, 97%, 99%, or 100% homology or identity with the sequence provided in FIG. 6 (SEQ ID NO: 1). Antibodies which bind the same epitope as an antibody provided herein are also encompassed by the instant invention.

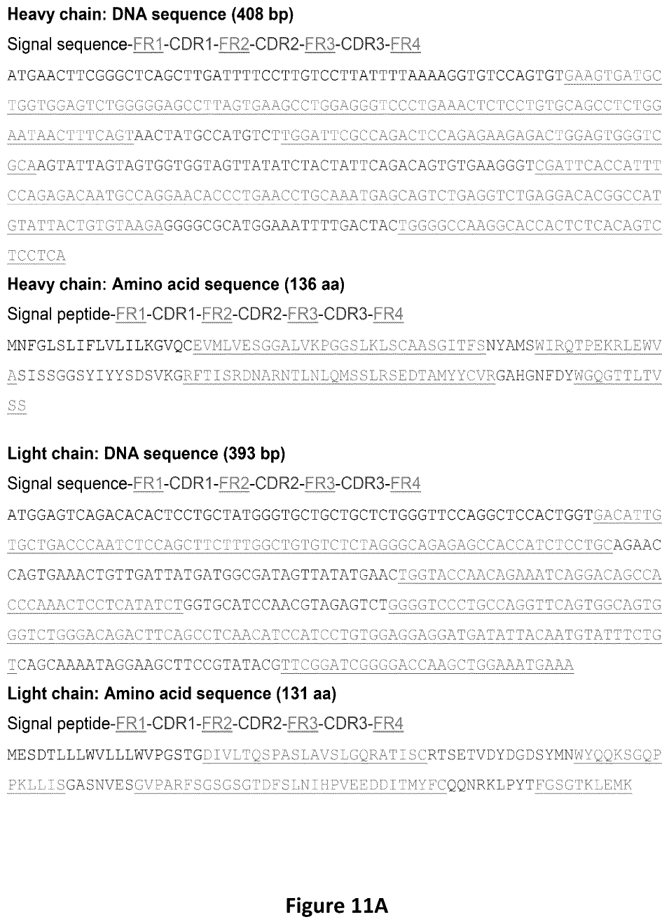

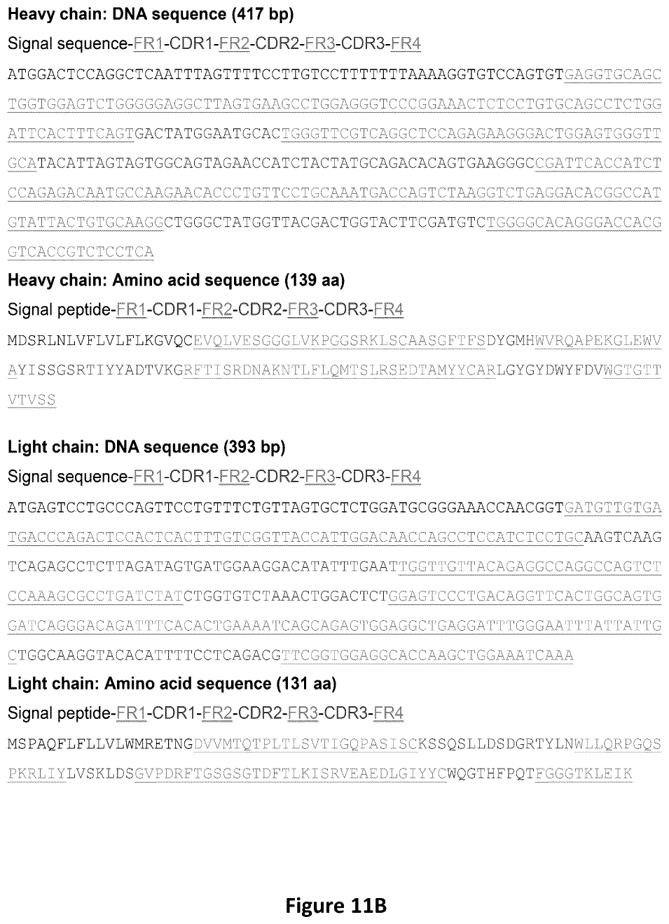

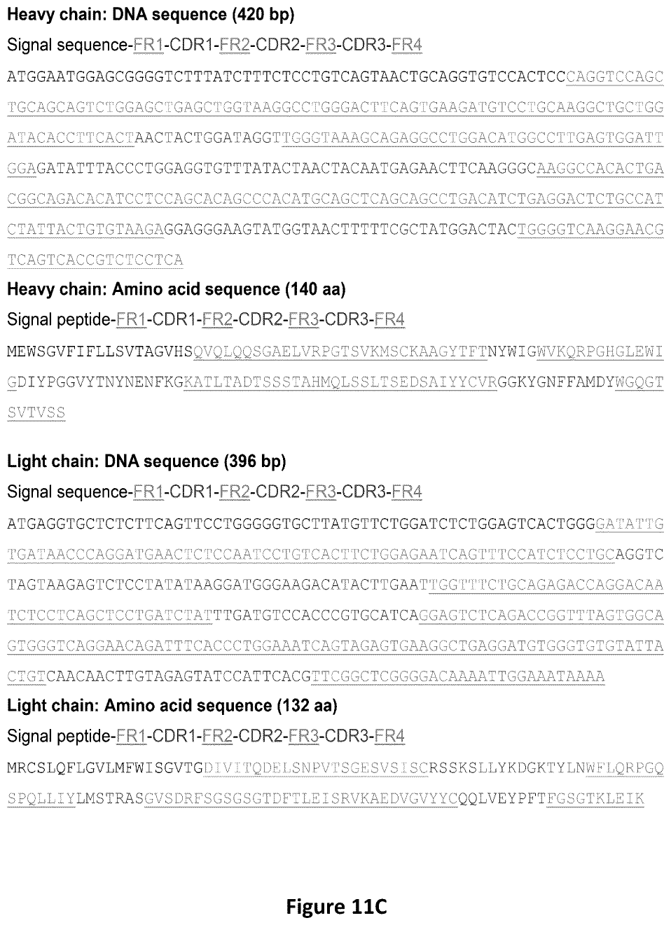

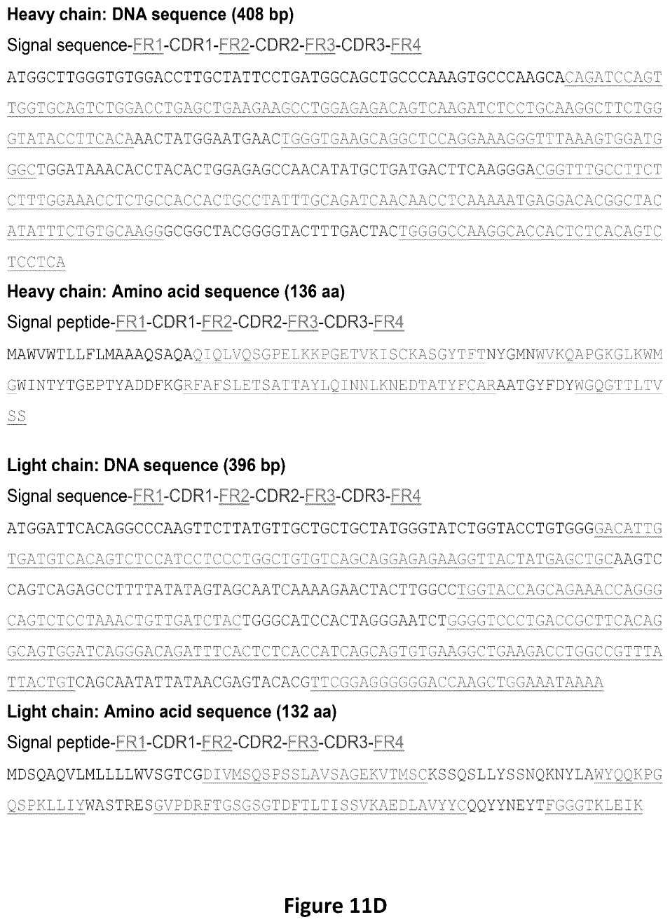

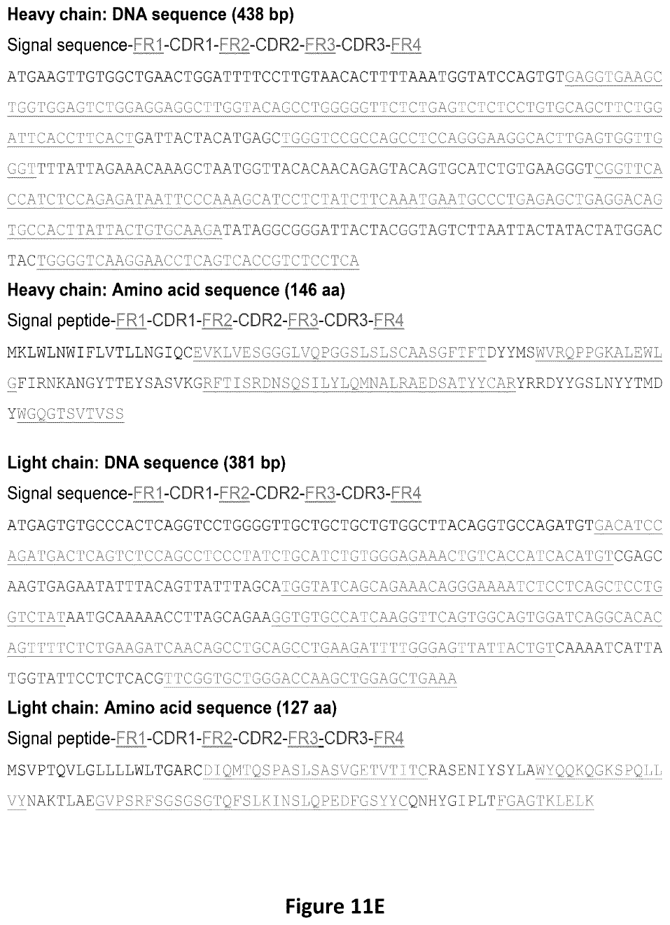

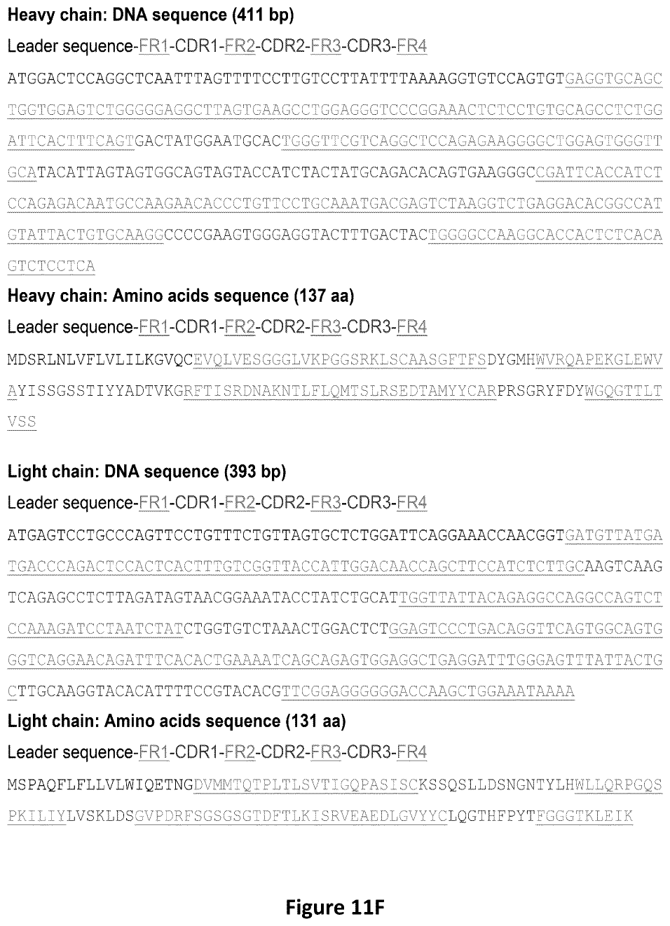

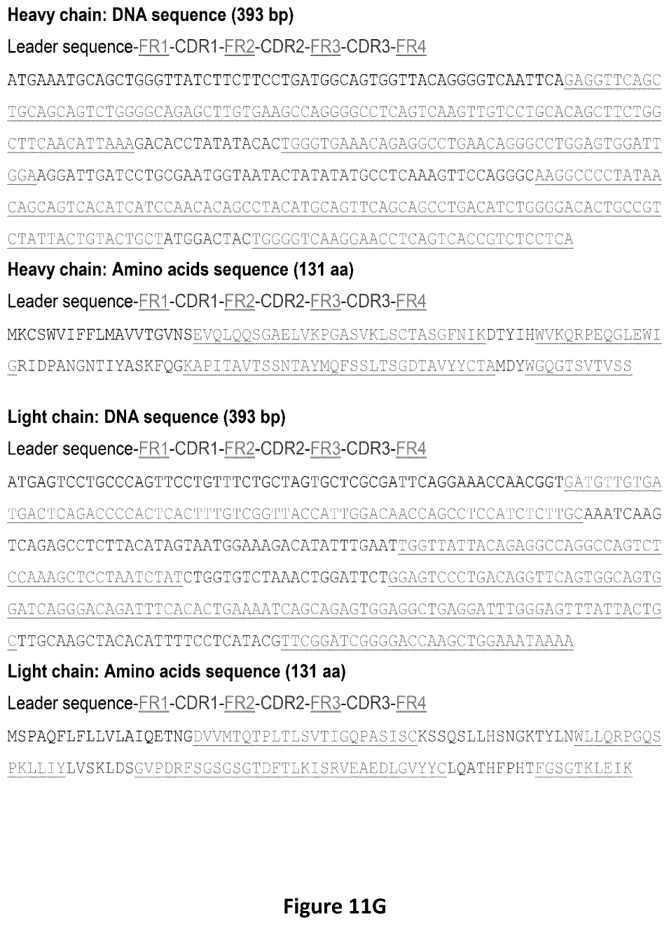

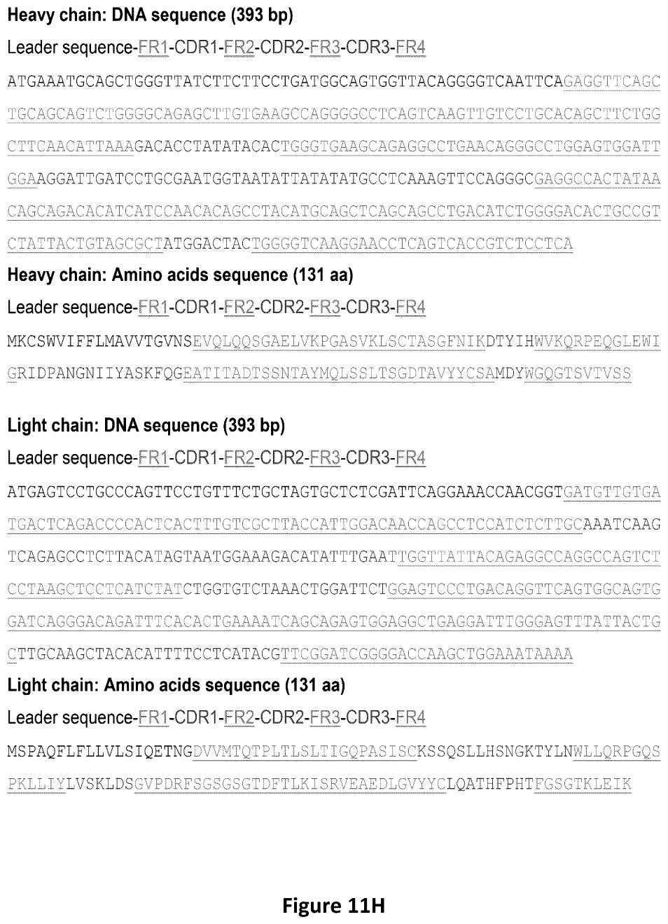

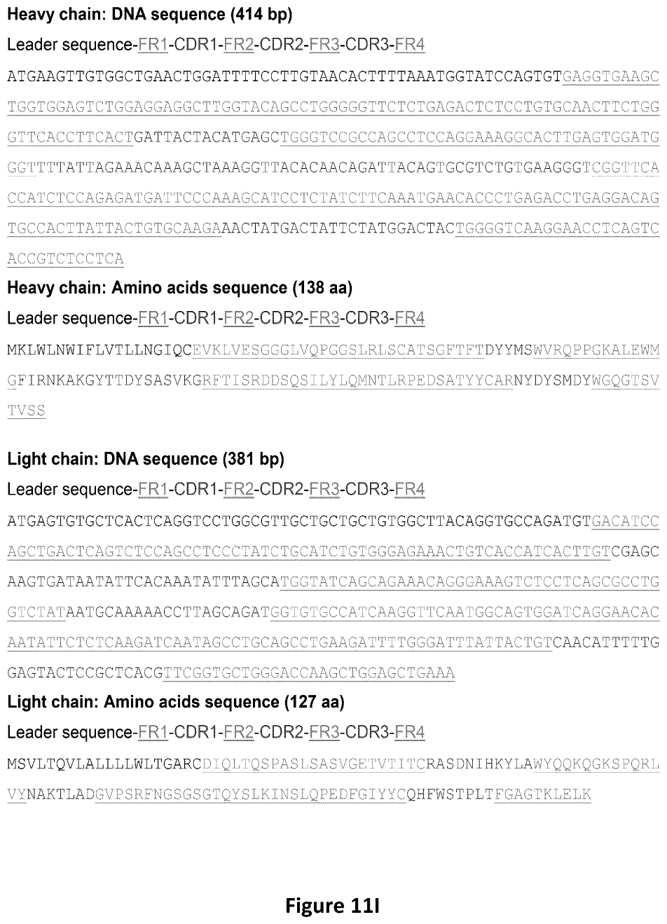

In a particular embodiment, the anti-TcfA antibody or fragment thereof is immunologically specific for amino acids 288-304 or 292-300 of TcfA. In a particular embodiment, the anti-TcfA antibody is 14D12 (as depicted in FIG. 11A), optionally wherein the signal peptides removed, or a fragment thereof. In a particular embodiment, the anti-TcfA antibody comprises a heavy chain comprising SEQ ID NO: 19 and/or a light chain comprising SEQ ID NO: 21. In a particular embodiment, the anti-TcfA antibody comprises a heavy chain comprising:

EVMLVESGGALVKPGGSLKLSCAASGITFSNYAMSWIRQTPEKRLEWV ASISSGGSYIYYSDSVKGRFTISRDNARNTLNLQMSSLRSEDTAMYYCVRGAH GNFDYWGQGTTLTVSS (SEQ ID NO: 22) and/or a light chain comprising: DIVLTQSPASLAVSLGQRATISCRTSETVDYDGDSYMNWYQQKSGQP PKLLISGASNVESGVPARFSGSGSGTDFSLNIHPVEEDDITMYFCQQNRKLPYT FGSGTKLEMK (SEQ ID NO: 23). In a particular embodiment, the anti-TcfA antibody or fragment thereof comprises one, two, three, four, five, or all six complementarity determining regions (CDRs) of 14D12 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11A. In a particular embodiment, the anti-TcfA antibody or fragment thereof comprises a heavy chain comprising one, two, or all three CDRs depicted in the heavy chain provided in FIG. 11A. In a particular embodiment, the anti-TcfA antibody or fragment thereof comprises a light chain comprising one, two, or all three CDRs depicted in the light chain provided in FIG. 11A. In a particular embodiment, the anti-TcfA antibody or fragment thereof comprises one, two, three, four, five, or all six of: NYAMS (SEQ ID NO: 24), SISSGGSYIYYSDSVKG (SEQ ID NO: 25), GAHGNFDY (SEQ ID NO: 26), RTSETVDYDGDSYMN (SEQ ID NO: 27), GASNVES (SEQ ID NO: 28), and QQNRKLPYT (SEQ ID NO: 29). In a particular embodiment, the anti-TcfA antibody or fragment thereof comprises a heavy chain comprising one, two, or all three of: NYAMS (SEQ ID NO: 24), SISSGGSYIYYSDSVKG (SEQ ID NO: 25), and GAHGNFDY (SEQ ID NO: 26) and/or a light chain comprising one, two, or all three of: RTSETVDYDGDSYMN (SEQ ID NO: 27), GASNVES (SEQ ID NO: 28), and QQNRKLPYT (SEQ ID NO: 29). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 19 and 22-29).

In a particular embodiment, the anti-TcfA antibody or fragment thereof is immunologically specific for amino acids 288-304 or 292-300 of TcfA. In a particular embodiment, the anti-TcfA antibody is 23F8 (as depicted in FIG. 11B), optionally wherein the signal peptides removed, or a fragment thereof. In a particular embodiment, the anti-TcfA antibody comprises a heavy chain comprising SEQ ID NO: 31 and/or a light chain comprising SEQ ID NO: 33. In a particular embodiment, the anti-TcfA antibody comprises a heavy chain comprising:

EVQLVESGGGLVKPGGSRKLSCAASGFTFSDYGMHWVRQAPEKGLEWV AYISSGSRTIYYADTVKGRFTISRDNAKNTLFLQMTSLRSEDTAMYYCARLGY GYDWYFDVWGTGTTVTVSS (SEQ ID NO: 34) and/or a light chain comprising:
DVVMTQTPLTLSVTIGQPASISCKSSQSLLDSDGRTYLNWLLQRPGQS PKRLIYLVSKLDSGVPDRFTGSGSGTDFTLKISRVEAEDLGIYYCWQGTHFPQ TFGGGTKLEIK (SEQ ID NO: 35). In a particular embodiment, the anti-TcfA antibody or fragment thereof comprises one, two, three, four, five, or all six CDRs of 23F8 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11B. In a particular embodiment, the anti-TcfA antibody or fragment thereof comprises a heavy chain comprising one, two, or all three CDRs depicted in the heavy chain provided in FIG. 11B. In a particular embodiment, the anti-TcfA antibody or fragment thereof comprises a light chain comprising one, two, or all three CDRs depicted in the light chain provided in FIG. 11B. In a particular embodiment, the anti-TcfA antibody or fragment thereof comprises one, two, three, four, five, or all six of: DYGMH (SEQ ID NO: 36), YISSGSRTIYYADTVKG (SEQ ID NO: 37), LGYGYDWYFDV (SEQ ID NO: 38), KSSQSLLDSDGRTYLN (SEQ ID NO: 39), LVSKLDS (SEQ ID NO: 40), and WQGTHFPQT (SEQ ID NO: 41). In a particular embodiment, the anti-TcfA antibody or fragment thereof comprises a heavy chain comprising one, two, or all three of: DYGMH (SEQ ID NO: 36), YISSGSRTIYYADTVKG (SEQ ID NO: 37), and LGYGYDWYFDV (SEQ ID NO: 38) and/or a light chain comprising one, two, or all three of: KSSQSLLDSDGRTYLN (SEQ ID NO: 39), LVSKLDS (SEQ ID NO: 40), and WQGTHFPQT (SEQ ID NO: 41). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 31 and 33-41).

In a particular embodiment, the anti-TcfA antibody or fragment thereof is immunologically specific for amino acids 337-345 of TcfA. In a particular embodiment, the anti-TcfA antibody is 18B2 (as depicted in FIG. 11C), optionally wherein the signal peptides removed, or a fragment thereof. In a particular embodiment, the anti-TcfA antibody comprises a heavy chain comprising SEQ ID NO: 43 and/or a light chain comprising SEQ ID NO: 45. In a particular embodiment, the anti-TcfA antibody comprises a heavy chain comprising:

QVQLQQSGAELVRPGTSVKMSCKAAGYTFTNYWIGWVKQRPGHGLEWI GDIYPGGVYTNYNENFKGKATLTADTSSSTAHMQLSSLTSEDSAIYYCVRGG KYGNFFAMDYWGQGTSVTVSS (SEQ ID NO: 46) and/or a light chain comprising:
DIVITQDELSNPVTSGESVSISCRSSKSLLYKDGKTYLNWFLQRPGQ SPQLLIYLMSTRASGVSDRFSGSGSGTDFTLEISRVKAEDVGVYYCQQLVEYP FTFGSGTKLEIK (SEQ ID NO: 47). In a particular embodiment, the anti-TcfA antibody or fragment thereof comprises one, two, three, four, five, or all six CDRs of 18B2 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11C. In a particular embodiment, the anti-TcfA antibody or fragment thereof comprises a heavy chain comprising one, two, or all three CDRs depicted in the heavy chain provided in FIG. 11C. In a particular embodiment, the anti-TcfA antibody or fragment thereof comprises a light chain comprising one, two, or all three CDRs depicted in the light chain provided in FIG. 11C. In a particular embodiment, the anti-TcfA antibody or fragment thereof comprises one, two, three, four, five, or all six of: NYWIG (SEQ ID NO: 48), DIYPGGVYTNYNENFKG (SEQ ID NO: 49), GGKYGNFFAMDY (SEQ ID NO: 50), RSSKSLLYKDGKTYLN (SEQ ID NO: 51), LMSTRAS (SEQ ID NO: 52), and QQLVEYPFT (SEQ ID NO: 53). In a particular embodiment, the anti-TcfA antibody or fragment thereof comprises a heavy chain comprising one, two, or all three of: NYWIG (SEQ ID NO: 48), DIYPGGVYTNYNENFKG (SEQ ID NO: 49), and GGKYGNFFAMDY (SEQ ID NO: 50) and/or a light chain comprising one, two, or all three of: RSSKSLLYKDGKTYLN (SEQ ID NO: 51), LMSTRAS (SEQ ID NO: 52), and QQLVEYPFT (SEQ ID NO: 53). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 43 and 45-53).

In a particular embodiment, the anti-TcfA antibody or fragment thereof is immunologically specific for amino acids 305-323 or 307-315 of TcfA. In a particular embodiment, the anti-TcfA antibody is 20F4 (as depicted in FIG. 11D), optionally wherein the signal peptides removed, or a fragment thereof In a particular embodiment, the anti-TcfA antibody comprises a heavy chain comprising SEQ ID NO: 55 and/or a light chain comprising SEQ ID NO: 57. In a particular embodiment, the anti-TcfA antibody comprises a heavy chain comprising:

QIQLVQSGPELKKPGETVKISCKASGYTFTNYGMNWVKQAPGKGLKWM GWINTYTGEPTYADDFKGRFAFSLETSATTAYLQINNLKNEDTATYFCARAA TGYFDYWGQGTTLTVSS (SEQ ID NO: 58) and/or a light chain comprising:
DIVMSQSPSSLAVSAGEKVTMSCKSSQSLLYSSNQKNYLAWYQQKPG QSPKWYWASTRESGVPDRFTGSGSGTDFTLTISSVKAEDLAVYYCQQYYNE YTFGGGTKLEIK (SEQ ID NO: 59). In a particular embodiment, the anti-TcfA antibody or fragment thereof comprises one, two, three, four, five, or all six CDRs of 20F4 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11D. In a particular embodiment, the anti-TcfA antibody or fragment thereof comprises a heavy chain comprising one, two, or all three CDRs depicted in the heavy chain provided in FIG. 11D. In a particular embodiment, the anti-TcfA antibody or fragment thereof comprises a light chain comprising one, two, or all three CDRs depicted in the light chain provided in FIG. 11D. In a particular embodiment, the anti-TcfA antibody or fragment thereof comprises one, two, three, four, five, or all six of: NYGMN (SEQ ID NO: 60), WINTYTGEPTYADDFKG (SEQ ID NO: 61), AATGYFDY (SEQ ID NO: 62), KSSQSLLYSSNQKNYLA (SEQ ID NO: 63), WASTRES (SEQ ID NO: 64), and QQYYNEYT (SEQ ID NO: 65). In a particular embodiment, the anti-TcfA antibody or fragment thereof comprises a heavy chain comprising one, two, or all three of: NYGMN (SEQ ID NO: 60), WINTYTGEPTYADDFKG (SEQ ID NO: 61), and AATGYFDY (SEQ ID NO: 62) and/or a light chain comprising one, two, or all three of: KSSQSLLYSSNQKNYLA

(SEQ ID NO: 63), WASTRES (SEQ ID NO: 64), and QQYYNEYT (SEQ ID NO: 65). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 55 and 57-65).

In a particular embodiment, the anti-TcfA antibody or fragment thereof is immunologically specific for amino acids 305-323 or 313-321 of TcfA. In a particular embodiment, the anti-TcfA antibody is 14G11 (as depicted in FIG. 11E), optionally wherein the signal peptides removed, or a fragment thereof In a particular embodiment, the anti-TcfA antibody comprises a heavy chain comprising SEQ ID NO: 67 and/or a light chain comprising SEQ ID NO: 69. In a particular embodiment, the anti-TcfA antibody comprises a heavy chain comprising:

EVKLVESGGGLVQPGGSLSLSCAASGFTFTDYYMSWVRQPPGKALEWL GFIRNKANGYTTEYSASVKGRFTISRDNSQSILYLQMNALRAEDSATYYCARY RRDYYGSLNYYTMD YWGQGTSVTVSS (SEQ ID NO: 70) and/or a light chain comprising:
DIQMTQSPASLSASVGETVTITCRASENIYSYLAWYQQKQGKSPQLL VYNAKTLAEGVPSRFSGSGSGTQFSLKINSLQPEDFGSYYCQNHYGIPLTFGA GTKLELK (SEQ ID NO: 71). In a particular embodiment, the anti-TcfA antibody or fragment thereof comprises one, two, three, four, five, or all six CDRs of 14G11 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11E. In a particular embodiment, the anti-TcfA antibody or fragment thereof comprises a heavy chain comprising one, two, or all three CDRs depicted in the heavy chain provided in FIG. 11E. In a particular embodiment, the anti-TcfA antibody or fragment thereof comprises a light chain comprising one, two, or all three CDRs depicted in the light chain provided in FIG. 11E. In a particular embodiment, the anti-TcfA antibody or fragment thereof comprises one, two, three, four, five, or all six of: DYYMS (SEQ ID NO: 72), FIRNKANGYTTEYSASVKG (SEQ ID NO: 73), YRRDYYGSLNYYTMDY (SEQ ID NO: 74), RASENIYSYLA (SEQ ID NO: 75), NAKTLAE (SEQ ID NO: 76), and QNHYGIPLT (SEQ ID NO: 77). In a particular embodiment, the anti-TcfA antibody or fragment thereof comprises a heavy chain comprising one, two, or all three of: DYYMS (SEQ ID NO: 72), FIRNKANGYTTEYSASVKG (SEQ ID NO: 73), and YRRDYYGSLNYYTMDY (SEQ ID NO: 74) and/or a light chain comprising one, two, or all three of: RASENIYSYLA (SEQ ID NO: 75), NAKTLAE (SEQ ID NO: 76), and QNHYGIPLT (SEQ ID NO: 77). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 67 and 69-77).

In a particular embodiment, the anti-TcfA antibody or fragment thereof is immunologically specific for amino acids 288-323, 229-240, 289-300, and/or 304-312 of TcfA. In a particular embodiment, the anti-TcfA antibody is 13E11 (as depicted in FIG. 11F), optionally wherein the signal peptides removed, or a fragment thereof. In a particular embodiment, the anti-TcfA antibody comprises a heavy chain comprising SEQ ID NO: 79 and/or a light chain comprising SEQ ID NO: 81. In a particular embodiment, the anti-TcfA antibody comprises a heavy chain comprising:

EVQLVESGGGLVKPGGSRKLSCAASGFTFSDYGMHWVRQAPEKGLEWV AYISSGSSTIYYADTVKGRFTISRDNAKNTLFLQMTSLRSEDTAMYYCARPRS GRYFDYWGQGTTLTVSS (SEQ ID NO: 82) and/or a light chain comprising:
DVMMTQTPLTLSVTIGQPASISCKSSQSLLDSNGNTYLHWLLQRPGQS PKILIYLVSKLDSGVPDRFSGSGSGTDFTLKISRVEAEDLGVYYCLQGTHFPYT FGGGTKLEIK (SEQ ID NO: 83). In a particular embodiment, the anti-TcfA antibody or fragment thereof comprises one, two, three, four, five, or all six CDRs of 13E11 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11F. In a particular embodiment, the anti-TcfA antibody or fragment thereof comprises a heavy chain comprising one, two, or all three CDRs depicted in the heavy chain provided in FIG. 11F. In a particular embodiment, the anti-TcfA antibody or fragment thereof comprises a light chain comprising one, two, or all three CDRs depicted in the light chain provided in FIG. 11F. In a particular embodiment, the anti-TcfA antibody or fragment thereof comprises one, two, three, four, five, or all six of: DYGMH (SEQ ID NO: 84), YISSGSSTIYYADTVKG (SEQ ID NO: 85), PRSGRYFDY (SEQ ID NO: 86), KSSQSLLDSNGNTYLH (SEQ ID NO: 87), LVSKLDS (SEQ ID NO: 88), and LQGTHFPYT (SEQ ID NO: 89). In a particular embodiment, the anti-TcfA antibody or fragment thereof comprises a heavy chain comprising one, two, or all three of: DYGMH (SEQ ID NO: 84), YISSGSSTIYYADTVKG (SEQ ID NO: 85), and PRSGRYFDY (SEQ ID NO: 86) and/or a light chain comprising one, two, or all three of: KSSQSLLDSNGNTYLH (SEQ ID NO: 87), LVSKLDS (SEQ ID NO: 88), and LQGTHFPYT (SEQ ID NO: 89). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 79 and 81-89).

In a particular embodiment, the anti-TcfA antibody or fragment thereof is immunologically specific for amino acids 140-160 or 139-150 of TcfA. In a particular embodiment, the anti-TcfA antibody is 10B 1 (as depicted in FIG. 11G), optionally wherein the signal peptides removed, or a fragment thereof In a particular embodiment, the anti-TcfA antibody comprises a heavy chain comprising SEQ ID NO: 91 and/or a light chain comprising SEQ ID NO: 93. In a particular embodiment, the anti-TcfA antibody comprises a heavy chain comprising:

EVQLQQSGAELVKPGASVKLSCTASGFNIKDTYIHWVKQRPEQGLEWI GRIDPANGNTIYASKFQGKAPITAVTSSNTAYMQFSSLTSGDTAVYYCTAMD YWGQGTSVTVSS (SEQ ID NO: 94) and/or a light chain comprising:
DVVMTQTPLTLSVTIGQPASISCKSSQSLLHSNGKTYLNWLLQRPGQS PKLLIYLVSKLDSGVPDRFSGSGSGTDFTLKISRVEAEDLGVYYCLQATHFPHT FGSGTKLEIK (SEQ ID NO: 95). In a particular embodiment, the anti-TcfA antibody or fragment thereof comprises one, two, three, four, five, or all six CDRs of 10B1 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11G. In a particular embodiment, the anti-TcfA antibody or fragment thereof comprises a heavy chain comprising one, two, or all three CDRs depicted in the heavy chain provided in FIG. 11G. In a particular embodiment, the anti-TcfA antibody or fragment thereof comprises a light chain comprising one, two, or all three CDRs depicted in the light chain provided in FIG. 11G. In a particular embodiment, the anti-TcfA antibody or fragment thereof comprises one, two, three, four, five, or all six of: DTYIH (SEQ ID NO: 96), RIDPANGNTIYASKFQG (SEQ ID NO: 97), MDY, KSSQSLLHSNGKTYLN (SEQ ID NO: 99), LVSKLDS (SEQ ID NO: 100), and LQATHFPHT (SEQ ID NO: 101). In a particular embodiment, the anti-TcfA antibody or fragment thereof comprises a heavy chain comprising one, two, or all three of: DTYIH (SEQ ID NO: 96), RIDPANGNTIYASKFQG (SEQ ID NO: 97), and MDY and/or a light chain comprising one, two, or all three of: KSSQSLLHSNGKTYLN (SEQ ID NO: 99), LVSKLDS (SEQ ID NO: 100), and LQATHFPHT (SEQ ID NO: 101). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 91 and 93-97, MDY, and 99-101).

In a particular embodiment, the anti-TcfA antibody or fragment thereof is immunologically specific for amino acids 140-160 or 139-150 of TcfA. In a particular embodiment, the anti-TcfA antibody is 7E11 (as depicted in FIG. 11H), optionally wherein the signal peptides removed, or a fragment thereof In a particular embodiment, the anti-TcfA antibody comprises a heavy chain comprising SEQ ID NO: 103 and/or a light chain comprising SEQ ID NO: 105. In a particular embodiment, the anti-TcfA antibody comprises a heavy chain comprising:

EVQLQQSGAELVKPGASVKLSCTASGFNIKDTYIHWVKQRPEQGLEWI GRIDPANGNIIYASKFQGEATITADTSSNTAYMQLSSLTSGDTAVYYCSAMDY WGQGTSVTVSS (SEQ ID NO: 106) and/or a light chain comprising:
DVVMTQTPLTLSLTIGQPASISCKSSQSLLHSNGKTYLNWLLQRPGQS PKLLIYLVSKLDSGVPDRFSGSGSGTDFTLKISRVEAEDLGVYYCLQATHFPHT FGSGTKLEIK (SEQ ID NO: 107). In a particular embodiment, the anti-TcfA antibody or fragment thereof comprises one, two, three, four, five, or all six CDRs of 7E11 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11H. In a particular embodiment, the anti-TcfA antibody or fragment thereof comprises a heavy chain comprising one, two, or all three CDRs depicted in the heavy chain provided in FIG. 11H. In a particular embodiment, the anti-TcfA antibody or fragment thereof comprises a light chain comprising one, two, or all three CDRs depicted in the light chain provided in FIG. 11H. In a particular embodiment, the anti-TcfA antibody or fragment thereof comprises one, two, three, four, five, or all six of: DTYIH (SEQ ID NO: 108), RIDPANGNIIYASKFQG (SEQ ID NO: 109), MDY, KSSQSLLHSNGKTYLN (SEQ ID NO: 111), LVSKLDS (SEQ ID NO: 112), and LQATHFPHT (SEQ ID NO: 113). In a particular embodiment, the anti-TcfA antibody or fragment thereof comprises a heavy chain comprising one, two, or all three of: DTYIH (SEQ ID NO: 108), RIDPANGNIIYASKFQG (SEQ ID NO: 109), and MDY and/or a light chain comprising one, two, or all three of: KSSQSLLHSNGKTYLN (SEQ ID NO: 111), LVSKLDS (SEQ ID NO: 112), and LQATHFPHT (SEQ ID NO: 113). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 103 and 105-109, MDY, and 111-113).

In a particular embodiment, the anti-TcfA antibody or fragment thereof is immunologically specific for amino acids 140-160 or 151-156 of TcfA. In a particular embodiment, the anti-TcfA antibody is 3E6 (as depicted in FIG. 11I), optionally wherein the signal peptides removed, or a fragment thereof. In a particular embodiment, the anti-TcfA antibody comprises a heavy chain comprising SEQ ID NO: 115 and/or a light chain comprising SEQ ID NO: 117. In a particular embodiment, the anti-TcfA antibody comprises a heavy chain comprising:

EVKLVESGGGLVQPGGSLRLSCATSGFTFTDYYMSWVRQPPGKALEWM GFIRNKAKGYTTDYSASVKGRFTISRDDSQSILYLQMNTLRPEDSATYYCARN YDYSMDYWGQGTSVTVSS (SEQ ID NO: 118) and/or a light chain comprising:
DIQLTQSPASLSASVGETVTITCRASDNIHKYLAWYQQKQGKSPQRL VYNAKTLADGVPSRFNGSGSGTQYSLKINSLQPEDFGIYYCQHFWSTPLTFGA GTKLELK (SEQ ID NO: 119). In a particular embodiment, the anti-TcfA antibody or fragment thereof comprises one, two, three, four, five, or all six CDRs of 3E6 (e.g., as determined by IMGT, Chothia, Kabat, Martin (e.g., enhanced Chothia) or AHo numbering scheme, particularly Kabat). In a particular embodiment, the anti-TcfA antibody or fragment thereof comprises one, two, three, four, five, or all six CDRs depicted in FIG. 11I. In a particular embodiment, the anti-TcfA antibody or fragment thereof comprises a heavy chain comprising one, two, or all three CDRs depicted in the heavy chain provided in FIG. 11I. In a particular embodiment, the anti-TcfA antibody or fragment thereof comprises a light chain comprising one, two, or all three CDRs depicted in the light chain provided in FIG. 11I. In a particular embodiment, the anti-TcfA antibody or fragment thereof comprises one, two, three, four, five, or all six of: DYYMS (SEQ ID NO: 120), FIRNKAKGYTTDYSASVKG (SEQ ID NO: 121), NYDYSMDY (SEQ ID NO: 122), RASDNIHKYLA (SEQ ID NO: 123), NAKTLAD (SEQ ID NO: 124), and QHFWSTPLT (SEQ ID NO: 125). In a particular embodiment, the anti-TcfA antibody or fragment thereof comprises a heavy chain comprising one, two, or all three of: DYYMS (SEQ ID NO: 120), FIRNKAKGYTTDYSASVKG (SEQ ID NO: 121), and NYDYSMDY (SEQ ID NO: 122) and/or a light chain comprising one, two, or all three of: RASDNIHKYLA (SEQ ID NO: 123), NAKTLAD (SEQ ID NO: 124), and QHFWSTPLT (SEQ ID NO: 125). In another embodiment, the anti-TcfA antibody or fragment thereof comprise an amino acid sequence having at least 90%, 95%, 97%, 99%, or 100% homology or identity with any of the sequences provided above (e.g., any of SEQ ID NOs: 115 and 117-125).

Compositions comprising an anti-TcfA antibody or fragment thereof and a carrier such as a pharmaceutically acceptable carrier are also encompassed by the instant invention. In a particular embodiment, the composition comprises at least one anti-TcfA antibody or antibody fragment and at least one carrier (e.g., a pharmaceutically acceptable carrier).

Nucleic acid molecules encoding an anti-TcfA antibody or fragment thereof are encompassed by the instant invention. Examples of nucleic acid molecules encoding anti-TcfA antibodies are provided in FIG. 11. In a particular embodiment, the nucleic acid molecule encoding the anti-TcfA antibody or fragment thereof comprise a nucleotide sequence having at least 90%, 95%, 97%, 99%, or 100% homology or identity with any of the sequences provided above (e.g., any of SEQ ID NOs: 18, 20, 30, 32, 42, 44, 54, 56, 66, 68, 78, 80, 90, 92, 102, 104, 114, and 116) or a nucleotide sequence encoding any of the amino sequences provided above. In a particular embodiment, the nucleic acid molecules of the instant invention are contained within a vector, particularly an expression vector. The instant invention also encompasses cells comprising and, optionally, expressing a nucleic acid molecule of the instant invention (e.g., hybridomas that secrete monoclonal anti-TcfA antibodies).

The antibody may be a synthetic or modified antibody (e.g., a recombinantly generated antibody; a chimeric antibody; a bispecific antibody; a humanized antibody; a camelid antibody; and the like). In a particular embodiment of the instant invention, the antibody is a monoclonal antibody.

The antibodies of the instant invention may be an antibody fragment. In a particular embodiment, the antibody fragment is an antigen binding fragment of the antibody. Antibody fragments include, without limitation, immunoglobulin fragments including, without limitation: single domain (Dab; e.g., single variable light or heavy chain domain), Fab, Fab′, F(ab′)₂, and F(v); and fusions (e.g., via a linker) of these immunoglobulin fragments including, without limitation: scFv, scFv₂, scFv-Fc, minibody, diabody, triabody, and tetrabody. The antibody may also be a protein (e.g., a fusion protein) comprising at least one antibody or antibody fragment.

The antibodies of the instant invention may be further modified. For example, the antibodies may be humanized. In a particular embodiment, the antibodies (or a portion thereof) are inserted into the backbone of an antibody or antibody fragment construct (e.g., an antibody framework), particularly a human construct/framework. For example, the variable light domain and/or variable heavy domain of the antibodies of the instant invention or the CDRs contained therein may be inserted into another antibody construct or framework, particularly human. Methods for recombinantly producing antibodies are well-known in the art. Indeed, commercial vectors for certain antibody and antibody fragment constructs are available.

The antibodies of the instant invention may also be conjugated/linked to other components. For example, the antibodies may be operably linked (e.g., covalently linked, optionally, through a linker) to at least one detectable agent, or imaging agent, contrast agent. Detectable agents include, without limitation, colloidal gold or gold nanoparticles, fluorescent probe, colored latex particles, colored cellulose nanobeads, horseradish peroxidase, and europium (Eu) nanoparticles. The antibodies of the instant invention may also comprise at least one purification tag (e.g., a His-tag). In a particular embodiment, the antibodies of the instant invention are conjugated to biotin or streptavidin (or analog thereof).

The antibody molecules of the invention may be prepared using a variety of methods known in the art. Polyclonal and monoclonal antibodies may be prepared as described in Current Protocols in Molecular Biology, Ausubel et al. eds. Antibodies may be prepared by chemical cross-linking, hybrid hybridoma techniques and by expression of recombinant antibody fragments expressed in host cells, such as bacteria or yeast cells. In one embodiment of the invention, the antibody molecules are produced by expression of recombinant antibody or antibody fragments in host cells. The nucleic acid molecules encoding the antibody may be inserted into expression vectors and introduced into host cells. The resulting antibody molecules are then isolated and purified from the expression system. The antibodies optionally comprise a purification tag by which the antibody can be purified.

The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

In accordance with another aspect of the instant invention, immunoassays for detecting B. pertussis are provided. In a particular embodiment, the immunoassay provides a rapid, point of care assay to detect B. pertussis during early disease. The immunoassays use at least one of the anti-TcfA antibodies of the instant invention. In a particular embodiment, the immunoassay can be performed at the point-of-care without the need for expensive equipment or the need for specialized equipment or off-site equipment to analyze the data. In a particular embodiment, the immunoassay is carried out using a sample capture device, such as a lateral flow device, more particularly a lateral flow test strip, that allows detection of the TcfA biomarker. In a particular embodiment, the immunoassay is performed on a lateral flow test strip.

The immunoassay of the instant invention has multiple advantages over existing diagnostics. For example, the immunoassay can be performed at the point of care. Other non-limiting advantages include low cost (e.g., no specialized equipment required), ease of use (e.g., no specialized user expertise required), and rapid results (e.g., less than about 40 minutes, less than about 30 minutes, less than about 20 minutes, or less than about 15 minutes). The immunoassay of the instant invention is also convenient for single samples and there is no need or advantage to batch samples.

Lateral flow immunoassays (LFI or LFIA) are simple tests for rapid detection of the presence or absence of a target analyte in a sample. Generally, lateral flow test strips comprise a matrix or material through which a mobile phase (e.g., a liquid sample) can flow through by capillary action to a test site where a detectable signal is generated to indicate the presence or absence of the target analyte. A lateral flow immunoassay may be used in a vertical or a horizontal orientation or in an orientation between vertical and horizontal.

A test strip is an article of manufacture that includes one or more zones, such as, for example, one or more of the following in any appropriate configurations: sample application area or sample pad, absorbent pad or wicking pad, test site, and conjugation pad. The different zones of the test strip may be made of the same material or different material. In a particular embodiment, the test site comprises nitrocellulose, nylon, polyester or polyester composite, matrix membranes (e.g., FUSION 5 (GE Healthcare Life Sciences, Pittsburgh, PA)), glass fiber membranes (e.g., Standard 14 or 17), or PVDF. In a particular embodiment, the test and control lines are on nitrocellulose. In a particular embodiment, the conjugate antibody is on a matrix membrane such as FUSION 5. In a particular embodiment, the sample application area or sample pad is a glass fiber membrane. The different zones may be joined by abutting and/or overlapping. The test strip may further comprise a backing to provide rigidity to the test strip (e.g., a supporting non-interactive material such as polyester).

Generally, the lateral flow immunoassay test strip of the present invention comprises a flow path from an upstream sample application area or sample pad to a test site or capture zone. The test site comprises an area (e.g., a line or stripe) of immobilized anti-TcfA antibodies (e.g., one or more anti-TcfA antibodies (either same or different epitopes)) and, optionally, a control area (e.g., a line or stripe) of immobilized control antibodies (e.g., anti-IgG antibodies, anti-species antibodies (e.g., anti-chicken IgG antibodies (e.g., from donkey or goat)). If the conjugated antibodies are biotin-labeled, then the control area (e.g., a line or stripe) may comprise streptavidin (or analogs thereof) instead of or in conjunction with the control antibodies. Alternatively, if the conjugated antibodies are labeled with streptavidin (or analogs thereof), then the control area (e.g., a line or stripe) may comprise biotin instead of or in conjunction with the control antibodies. When present, the control line is preferably further downstream than the test line. Downstream of the test site is generally an absorbent pad or wicking pad to promote capillary action and movement of the fluid from the sample application area or sample pad. Downstream of the sample application area or sample pad and upstream to the test site is a conjugation pad. The conjugation pad comprises anti-TcfA antibodies (e.g., one or more anti-TcfA antibodies (either same or different epitopes)) conjugated to a detectable agent (e.g., colloidal gold or gold nanoparticles, fluorescent probe, colored latex particles, colored cellulose nanobeads, horseradish peroxidase, and europium (Eu) nanoparticles). In a particular embodiment, the detectable agent generates a direct signal that can be observed by a human (e.g., color from gold colloidal). While other detectable agents require additional steps to produce a signal (e.g., an enzyme to produce detectable product upon reaction with suitable substrate (e.g., horseradish peroxidase); a secondary antibody, etc.), these detectable agents are less preferred.

The assay run length of the lateral flow immunoassay test strip will generally be less than 100 mm in length (e.g., including sample pad, conjugate pad, nitrocellulose, and wicking pad). In a particular embodiment, the assay run length is less than about 80 mm, less than about 70 mm, or less than about 60 mm in length. The test site need only be long enough (e.g., at least about 10 mm) to be able to visualize and differentiate the test line and the control line, when present.

Generally, the anti-TcfA antibody of the conjugation pad binds a different epitope than the anti-TcfA antibody of the test site. In a particular embodiment, the anti-TcfA antibody of the conjugation pad (e.g., the antibody conjugated to a detectable agent such as gold) binds amino acids 140-160 of TcfA. In a particular embodiment, the anti-TcfA antibody of the conjugation pad is 7E11, 10B1, or 3E6 (particularly 10B1) or an anti-TcfA antibody which is a fragment or homolog of 7E11, 10B1, or 3E6 as described hereinabove (e.g., an antibody which binds the same epitope or an antibody comprising all 6 CDRs of 7E11, 10B1, or 3E6). In a particular embodiment, the anti-TcfA antibody of the test site binds amino acids 288-304, amino acids 305-323, and/or amino acids 322-330 of TcfA. In a particular embodiment, the anti-TcfA antibody of the test site is 13E11, 14D12, 23F8, 19F9, 14D9, or 25E3 (particularly 13E11 and/or 14D12) or an anti-TcfA antibody which is a fragment or homolog of 13E11, 14D12, 23F8, 19F9, 14D9, or 25E3 as described hereinabove (e.g., an antibody which binds the same epitope or an antibody comprising all 6 CDRs of 13E11, 14D12, 23F8, 19F9, 14D9, or 25E3). In a particular embodiment, the anti-TcfA antibody of the conjugation pad is 10B1 or an anti-TcfA antibody which is a fragment or homolog of 10B1 as described hereinabove (e.g., an antibody which binds the same epitope as 10B1 or an antibody comprising all 6 CDRs of 10B1) and the test site comprises 1) 13E11 or an anti-TcfA antibody which is a fragment or homolog of 13E11 as described hereinabove (e.g., an antibody which binds the same epitope as 13E11 or an antibody comprising all 6 CDRs of 13E11), and 2) 14D12 or an anti-TcfA antibody which is a fragment or homolog of 14D12 as described hereinabove (e.g., an antibody which binds the same epitope as 14D12 or an antibody comprising all 6 CDRs of 14D12).

In general, a fluid sample (e.g., by dipping or spotting) is applied to the sample application area or sample pad. In a particular embodiment, the sample is a biological sample obtained from a subject. Biological samples obtained from the subject to be used in the immunoassay of the instant invention include, without limitation: nasopharyngeal swabs, aspirates, and washes. The cells in the biological sample may be lysed prior to analysis. The obtained biological sample may be diluted, purified, concentrated, filtered, dissolved, suspended or otherwise manipulated prior to assay to optimize the immunoassay results. In a particular embodiment, the biological sample is diluted in a carrier or buffer (e.g., a dilution and/or extraction buffer). In a particular embodiment, biological sample is diluted in phosphate buffered saline (PBS). In a particular embodiment, biological sample is diluted in PBS comprising a surfactant (e.g., 0.05% to 2.0%, particularly, 0.1%, 0.25%, 0.5%, 0.75%, or 1.0%), such as an anionic surfactant. Examples of surfactants include, without limitation, alkyl sulfates, alkyl ether sulfates, alkyl ether phosphates, ammonium lauryl sulfate, sodium dodecyl sulfate (SDS), sodium lauryl ether sulfate, and sodium myreth sulfate.

The biological sample for testing can be from any subject. The immunoassay will be particularly useful for the rapid diagnosis of infants (e.g., in pediatrician offices, urgent care clinics, small hospital emergency departments, or medical clinics) with early pertussis. Infants have higher bacterial loads than children or adults (Eby, et al., Infect. Immun. (2013) 81(5):1390-8; Nakamura et al. (2011) Clin. Microbiol. Infect. (2011) 17(3):365-70; Tenenbaum et al., Eur. J. Clin. Microbiol. Infect. Dis. (2012) 31(11):3173-82). This higher load can facilitate detection of B. pertussis antigens. Nonetheless, the immunoassay of the instant invention can also be used on older patients (e.g., children and adults).

Methods for detecting B. pertussis are provided using an immunoassay are also encompassed by the instant invention. In a particular embodiment, the method comprises obtaining a sample (e.g., a biological sample (e.g., from a subject)), diluting the sample in a carrier or buffer, and applying the diluted sample to the immunoassay (e.g., LFI). In a particular embodiment, the method further comprises diagnosing a subject as having a B. pertussis infection based on the presence of a positive result from the immunoassay.

The immunoassays of the instant invention may further comprise assays, particularly other point of care assays. In a particular embodiment, the pertussis diagnostic technology of the instant invention is combined or multiplexed with other respiratory diseases or disorders. In a particular embodiment, the immunoassays of the instant invention further comprise an assay for respiratory syncytial virus (RSV). Many RSV-positive infants are also pertussis-positive (generally 8-16%) and most infants with pertussis are co-infected with RSV (approximately 67-74%) (Nuolivirta, et al., Pediatr. Infect. Dis. J. (2010) 29(11):1013-5; Cosnes-Lambe, et al., Eur. J. Pediatr. (2008) 167(9):1017-9). There is no difference in cough symptoms at hospital admission between infants infected only with RSV vs. infants coinfected with RSV and pertussis (Nuolivirta, et al., Pediatr. Infect. Dis. J. (2010) 29(11):1013-5; Crowcroft, et al., Arch. Dis. Child. (2003) 88(9):802-6). But importantly, detecting co-infection is clinically relevant as infants with RSV and B. pertussis co-infections need antibiotics whereas infants infected solely with RSV do not antibiotics (Bronchiolitis AaoPSoDaMo, Pediatrics (2006) 118(4):1774-93). Thus, the instant invention also encompasses immunoassays, particularly LFIs, comprising the anti-TcfA antibodies of the instant invention in a first test line and RSV detecting agents in a second test line (e.g., antibodies against RSV viral fusion protein (see, e.g., QuickVue® RSV Test (Quidel, San Diego CA)) and/or RSV nucleotprotein (see, e.g., ImmunoCard STAT!® RSV (Meidian Biosciences, Cincinnati, OH)). These LFIs allow for rapid diagnosis of B. pertussis and/or RSV infections that could otherwise remain undiagnosed and untreated, particularly in infants.

Generally, the immunoassays of the instant invention will be used as a diagnostic for pertussis. Significantly, the immunoassays of the instant invention can also be used to monitor the therapy of a subject with pertussis. For example, after diagnosis with pertussis and administration of an appropriate therapy (e.g., antibiotics), the immunoassays of the instant invention can be used to monitor the amount of B. pertussis and the clearance of the bacteria.

While the present invention has been described with regard to pertussis diagnosis and detecting B. pertussis, the gene for TcfA is also found in B. parapertussis, B. holmesii, and B. bronchiseptica. Notably, B. parapertussis and B. holmesii infect humans and B. bronchiseptica is the causative agent for kennel cough in animals. Thus, in certain embodiments, the anti-TcfA antibodies can also be used to detect the presence of B. parapertussis, B. holmesii, and B. bronchiseptica. Indeed, as seen in Example 2, B. holmesii was detected with one of the five LFIs tested.

In a particular embodiment, the immunoassay of the instant invention is specific for diagnosis and detecting B. pertussis. For example, the immunoassay may be specific for diagnosis and detecting B. pertussis while not significantly detecting B. parapertussis, B. holmesii, and/or B. bronchiseptica.

In accordance with another aspect of the instant invention, compositions and methods for the inhibition, treatment, and/or prevention of pertussis and/or a B. pertussis infection are provided. The methods comprise administering an anti-TcfA antibody or fragment thereof of the instant invention to a subject in need thereof. The anti-TcfA antibodies may be administered in a composition further comprising a pharmaceutically acceptable carrier. The composition may further comprise at least one other therapeutic agent against B. pertussis (e.g., antibiotics). Alternatively, the other therapeutic agent may be contained within a separate composition(s) with at least one pharmaceutically acceptable carrier. The composition(s) comprising at least one anti-TcfA antibody and/or the composition(s) comprising at least one other therapeutic agent may be contained within a kit.

TcfA has been shown to be required for efficient colonization of the trachea in a mouse model of pertussis (Finn, et al., Mol. Microbiol. (1995) 16(4):625-634). Furthermore, the heterologous expression of TcfA in E. coli enables the engineered E. coli to bind human lung epithelial cells, thereby suggesting a role for TcfA for cellular binding and infection (Gasperini, et al., Mol. Cell Proteomics (2018) 17(2):205-215). Notably, passive immunity has been demonstrated with humanized mouse anti-pertussis toxin mAbs when administered to infected baboons. The anti-pertussis toxin mAbs were capable of blunting the post-infection white blood cell increase and increasing the speed by which B. pertussis bacteria was cleared (Nguyen, et al., Sci. Transl. Med. (2015) 7(316):316ra195). Based on the requirement for TcfA for efficient colonization, blocking TcfA with an antibody of the instant invention will provide therapeutic effects by inhibiting the ability of B. pertussis to bind and infect cells.

The compositions of the present invention can be administered by any suitable route, for example, by injection (e.g., for local or systemic administration), intravenous, oral, pulmonary, nasal or other modes of administration. In a particular embodiment, the compositions are administered orally or by inhalation. The compositions comprising the antibodies of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. Selection of a suitable pharmaceutical preparation depends upon the method of administration chosen. The concentration of the antibodies in the chosen medium may be varied and the medium may be chosen based on the desired route of administration of the pharmaceutical preparation. Except insofar as any conventional media or agent is incompatible with the agents to be administered, its use in the pharmaceutical preparation is contemplated. The pharmaceutical composition of the present invention can be prepared, for example, in liquid form, or can be in dried powder form (e.g., lyophilized).

Pharmaceutical compositions containing agents of the present invention as the active ingredient in intimate admixture with a pharmaceutical carrier can be prepared according to conventional pharmaceutical compounding techniques. The carrier may take a wide variety of forms depending on the form of preparation desired for administration. In preparing the antibody in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets).

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

The dose and dosage regimen of the antibodies according to the invention that is suitable for administration to a particular patient may be determined by a physician considering the patient's age, sex, weight, general medical condition, and the specific condition and severity thereof for which the agent is being administered. The physician may also consider the route of administration of the antibodies, the pharmaceutical carrier with which the antibodies may be combined, and the antibodies' biological activity. The appropriate dosage unit for the administration of the agents of the invention may be determined by evaluating the toxicity of the agents in animal models. Appropriate dosage unit may also be determined by assessing the efficacy of the agents in combination with other standard drugs.

The compositions comprising the agents of the instant invention may be administered at appropriate intervals, for example, at least once a day or more until the pathological symptoms are reduced or alleviated, after which the dosage may be reduced to a maintenance level. The appropriate interval in a particular case would normally depend on the condition of the patient.

### DEFINITIONS

The following definitions are provided to facilitate an understanding of the present invention:

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers. Suitable pharmaceutical carriers are described, for example, in “Remington's Pharmaceutical Sciences” by E. W. Martin.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule (e.g., antigen-binding fragment), and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, “diagnose” refers to detecting and identifying a disease or disorder in a subject. The term may also encompass assessing or evaluating the disease or disorder status (progression, regression, stabilization, response to treatment, etc.) in a patient known to have the disease or disorder.

As used herein, the term “prognosis” refers to providing information regarding the impact of the presence of a disease or disorder (e.g., as determined by the diagnostic methods of the present invention) on a subject's future health (e.g., expected morbidity or mortality). In other words, the term “prognosis” refers to providing a prediction of the probable course and outcome of a disease/disorder and/or the likelihood of recovery from the disease/disorder.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of a disease or disorder herein may refer to curing, relieving, and/or preventing the disease or disorder, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

The term “isolated” refers to the separation of a compound from other components present during its production or from its natural environment. “Isolated” is not meant to exclude artificial or synthetic mixtures with other compounds or materials, or the presence of impurities that do not substantially interfere with the fundamental activity, and that may be present, for example, due to incomplete purification, or the addition of stabilizers.

As used herein, a “biological sample” refers to a sample of biological material obtained from a subject, particularly a human subject, including a tissue, a tissue sample, cell(s), and a biological fluid (e.g., blood (e.g., whole blood), serum, plasma, urine, sweat, tears, saliva, mucosal secretions, sputum, CSF).

The following examples are provided to illustrate various embodiments of the present invention. The examples are not intended to limit the invention in any way.

### Example 1

A bioinformatics-based strategy was used to identify tracheal colonization factor A (TcfA) as a novel biomarker for B. pertussis infection. Epitope-specific polyclonal antibodies (pAbs) that recognize the cell-associated and secreted isoforms of TcfA were developed. Specifically, polyclonal antibodies were generated against amino acids 140-160, 288-304, or 305-323 of TcfA.

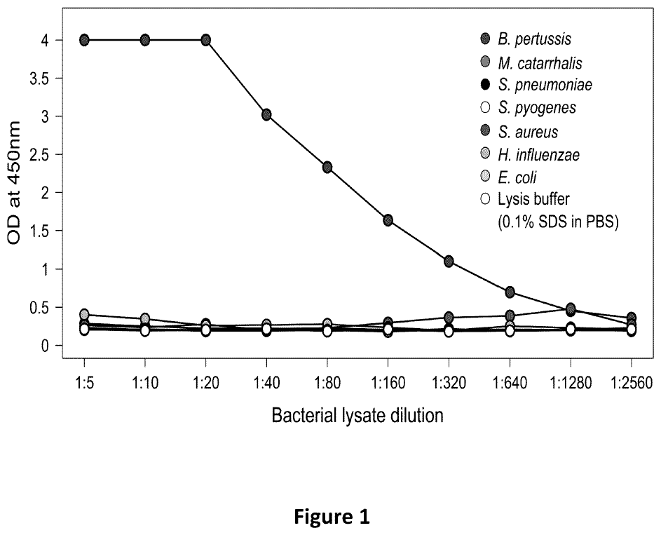

The specificity of the anti-TcfA antibodies was tested. Specifically, the anti-TcfA pAbs were incorporated into immunoassays, particularly in ELISA and LFI formats. Cultures of bacterial species potentially found in the nasopharynx (Bordetella pertussis, Moraxella catarrhalis, Streptococcus pneumoniae, Streptococcus pyogenes, Staphylococcus aureus, Haemophilus influenza, and Escherichia coli) were washed from culture plates with phosphate buffered saline (PBS), normalized to an OD 600 of 1.0, and lysed in 1% sodium dodecyl sulfate (SDS) in PBS for 1 hour at 65° C. The lysates were then tested by antigen-capture ELISA with anti-TcfA pAbs. As seen in FIG. 1, an antigen-capture ELISA with the anti-TcfA pAbs does not detect (i.e., cross-react) with any of the other tested bacteria. In addition to those shown in FIG. 1, the anti-TcfA pAb-based antigen-capture ELISA did not cross-react with Enterobacter aerogenes, Enterococcus faecalis, Staphylococcus epidermidis, Streptococcus mutans, Streptococcus mitis, and Corynebacterium pseudodiptherium. Similarly, a strong ability to detect B. pertussis lysates while showing no detectable cross-reactivity with the above bacteria was observed with the LFI immunoassay. Notably, when the pAbs are used individually in Western blot with lysates of these bacteria, some of them do react with a very limited number of protein bands in only some of the other bacterial species. However, antigen-capture ELISA and LFI require both the capture and detector antibodies to bind the same target. Therefore, it is unlikely that the same cross-reactive protein would be picked up by both the capture and detector pAb since the cross-reactivity in Western blot was so limited. Consequently, neither the antigen-capture ELISA nor the LFI resulted in the detection of any of the other listed bacterial species.

Despite demonstrating specificity for B. pertussis, the anti-TcfA pAb-based immunoassay was still able to recognize multiple strains of B. pertussis. Specifically, material from two B. pertussis strains—Tohama and 165—were tested. Both the Tohama and 165 strains were detected by all anti-TcfA pAb combinations tested by LFI. This result is likely due to the conserved epitopes of the anti-TcfA antibodies. Indeed, the peptide sequences of all 3 of the targeted TcfA epitopes are conserved in >99% (n=436) of clinical isolates from the U.S. and 8 other countries (van Amersfoorth, et al., J. Clin. Microbiol. (2005) 43(6):2837-43; Packard, et al., J. Med. Microbiol. (2004) 53(Pt 5):355-65; van Loo, et al., J. Clin. Microbiol. (2002) 40(6):1994-2001).

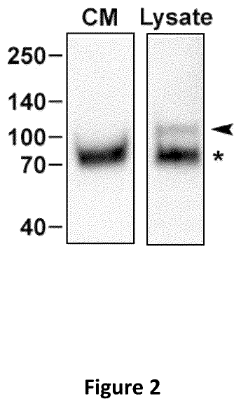

TcfA has a cell-associated and a secreted isoform (Finn, et al., Mol. Microbiol. (1995) 16(4):625-34), and the anti-TcfA pAbs described herein are reactive with both isoforms. As seen in FIG. 2, both isoforms were detectable in a Western blot assay as two bands of the appropriate molecular weight are clearly present in cellular lysates. Notably, only the secreted form is observed in 0.2 μm-filtered B. pertussis conditioned liquid medium (Stainer-Scholte medium). As with the Western blot analysis, both isoforms were detectable using a LFI assay. The secreted isoform was detected on 0.2 μm-filtered B. pertussis conditioned liquid medium (Stainer-Scholte medium) and the cell-associated isoform was detected on intact, formaldehyde-inactivated cells. Thus, sample preparation for analysis by LFI does not require cell lysis. However, it was determined that detergent lysis of B. pertussis cells increased the sensitivity of the anti-TcfA pAb-based LFI. Lysis in 0.1% SDS in PBS, pH 7.4 at room temperature for as little as 5 minutes provided an enhanced signal compared to unlysed samples, but more signal was seen after 60 minutes of lysis or when the lysis was performed at 65° C. rather than room temperature.

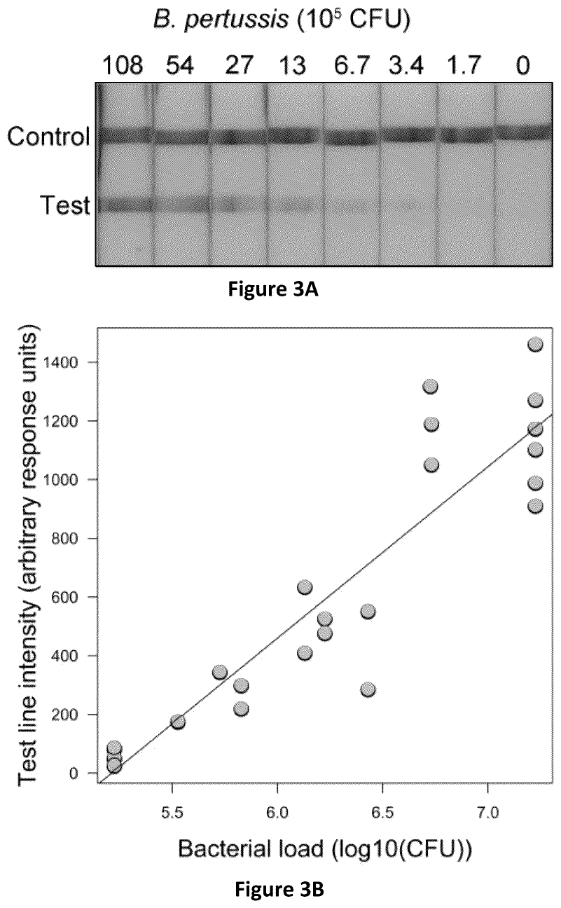

The limit of detection of with one of the anti-TcfA pAbs was tested. Specifically, an LFI was performed with B. pertussis lysates prepared by detergent lysis for 60 minutes at 65° C. with 0.1% SDS in PBS. The lowest serial dilution scored as positive by 3 of 3 blinded reviewers contained material from 1.7×10⁵ colony forming units (CFU) (FIG. 3A). A quantitative limit of detection analysis was also performed. The ESE-Quant lateral flow reader (Qiagen, Germantown, MD) and LF Studio software were used to quantify the test line intensity of multiple dilutions tested in replicates over three days. Background-adjusted data were fit to a linear curve on a semi-log plot with an R² value of 0.85 (FIG. 3B). The calculated limit of detection was 1.6×10⁵ CFU, which fits well with the limit of detection determined by reviewers visually. The limit of detection by antigen-capture ELISA was determined to be 3.3×10⁴ CFU. Notably, both the LFI and ELISA limits of detection are well below the bacterial burden found in infant nasopharynx washes (10⁷ to 10¹⁰ CFU/ml) or swabs (10⁶ CFU) (Eby, et al., Infect. Immun. (2013) 81(5):1390-8; Nakamura, et a., Clin. Microbiol. Infect. (2011) 17(3):365-70; Tenenbaum, et al., Eur. J. Clin. Microbiol. Infect. Dis. (2012) 31(11):3173-82).

### Example 2

Although pAbs can be used in commercial LFIs, monoclonal antibodies (mAbs) are preferred for commercialization because of their superior affinity (i.e., better LFI sensitivity), lot-to-lot reproducibility, and their more efficient, reliable, and economic large-scale production. Thus, a library of anti-TcfA mAbs was developed and the mAbs were incorporated into an advanced mAb-based LFI diagnostic to achieve enhanced analytical sensitivity and better commercialization potential. Generating a library of mAbs against TcfA required an unusually large number of immunization and hybridoma screening strategies. Ultimately, a large library of mAb-secreting hybridomas was generated and the mAbs showed robust reactivity in ELISA with immobilized antigen (recombinant TcfA, endogenous TcfA including formaldehyde-inactivated B. pertussis cells and/or TcfA peptides). Thirty-six mAbs were advanced for large-scale hybridoma culture and mAb purification via Protein A affinity chromatography.

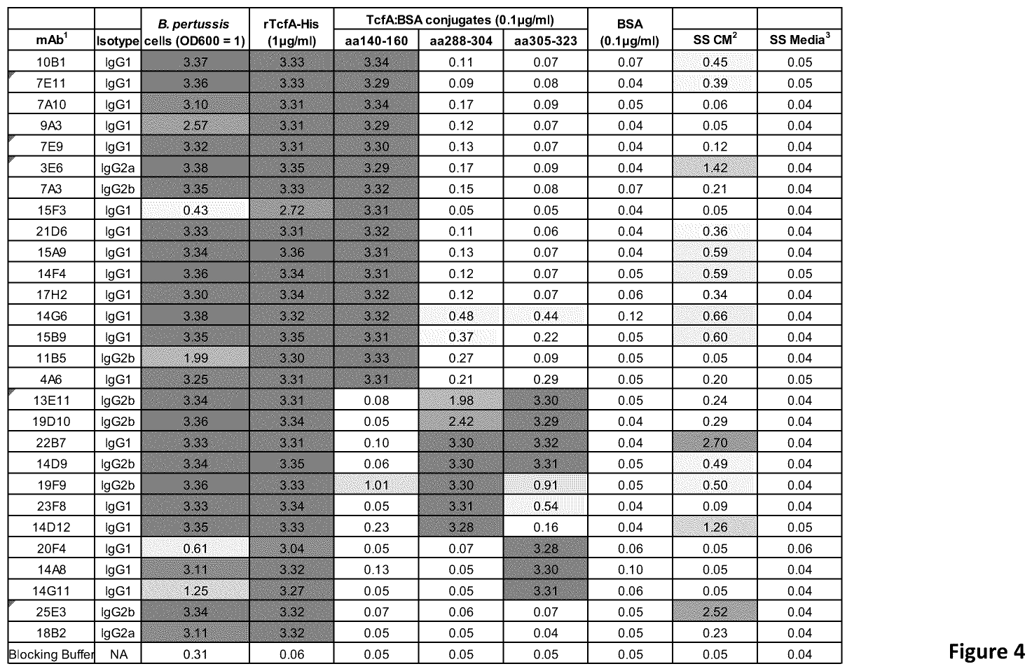

The reactivity of the mAbs was measured by indirect ELISA with plates coated with various antigen (FIG. 4). The immobilized antigens were formaldehyde-inactivated B. pertussis cells, recombinant TcfA containing a histidine tag, TcfA peptides (amino acids 140-160, 288-304, or 305-323) conjugated to bovine serum albumin (BSA), or control BSA. The reactivity of the mAbs was also tested against clarified, 0.2 μm-filtered supernatant from B. pertussis Tohama I cultures grown in Stainer-Scholte medium or Stainer-Scholte uninocculated medium. As seen in FIG. 4, most of the mAb demonstrated a strong but selective reactivity for TcfA.

In order to determine the epitopes recognized by the mAb, purified mAbs were evaluated by indirect ELISA with streptavidin plates loaded with biotinylated peptides. The peptides were 15 mers that were offset by 3 amino acids. The minimal overlapping peptide sequence for wells with an OD₄₅₀ greater than 0.75 (and in a series of two or more such adjacent wells) was defined as the minimal linear peptide epitope (Table 1). Notably, mAbs 22B7 and 14D9 reacted with a long series of peptides such that the first peptide in the series did not overlap with the last peptide. For these mAbs, the peptide sequence defined by the entire series of reactive wells is provided. In addition, purified mAbs were evaluated by indirect ELISA with plates coated with the TcfA protein fragments. With the exception of amino acids 40-374, all protein fragments were conjugated to bovine serum albumin.

TABLE 1
Epitopes within TcfA recognized by the mAbs.
The sequence identifiers for the provided minimal linear
peptide epitope are: aa139-150 (SEQ ID NO: 2); aa148-159 (SEQ
ID NO: 3); aa151-156 (SEQ ID NO: 4); aa151-159 (SEQ ID NO: 5);
aa229-240 (SEQ ID NO: 6); aa289-300 (SEQ ID NO: 7); aa304-312
(SEQ ID NO: 8); aa286-321 (SEQ ID NO: 9); aa289-324 (SEQ ID
NO: 10); aa289-294 (SEQ ID NO: 11); aa292-300 (SEQ ID NO: 12);
aa307-315 (SEQ ID NO: 13); aa3 10-315 (SEQ ID NO: 14); aa313-
321 (SEQ ID NO: 15); aa322-330 (SEQ ID NO: 16); and
aa337-345 (SEQ ID NO: 17).

| | | | Protein fragment |
|---|---|---|---|
| mAb | Minimal linear peptide epitope | | reactivity |
| 10B1 | aa139-150 | PGIGKVGGSAPG | aa140-160 |
| 7E11 | aa139-150 | PGIGKVGGSAPG | aa140-160 |
| 7A10 | aa148-159 | APGPDTSTGSGP | aa140-160 |
| 9A3 | aa148-159 | APGPDTSTGSGP | aa140-160 |
| 7E9 | aa148-159 | APGPDTSTGSGP | aa140-160 |
| 3E6 | aa151-156 | PDTSTG | aa140-160 |
| 7 A3 | aa151-156 | PDTSTG | aa140-160 |
| 15F3 | aa151-159 | PDTSTGSGP | aa140-160 |
| 21D6 | aa151-159 | PDTSTGSGP | aa140-160 |
| 15A9 | aa151-159 | PDTSTGSGP | aa140-160 |
| 14F4 | aa151-159 | PDTSTGSGP | aa140-160 |
| 17H2 | aa151-159 | PDTSTGSGP | aa140-160 |
| 14G6 | aa151-159 | PDTSTGSGP | aa140-160 |
| 15B9 | aa151-159 | PDTSTGSGP | aa140-160 |
| 11B5 | aa151-159 | PDTSTGSGP | aa140-160 |
| 4A6 | aa151-159 | PDTSTGSGP | aa140-160 |
| 13E11 | aa229-240; | PADGGQDGPPPP; | aa288-304, |
| | aa289-300; | LPERGDDAGPKP; | aa305-323 |
| | aa304-312 | EGGDEGPQP | |
| 19D10 | aa229-240; | PADGGQDGPPPP; | aa288-304, |
| | aa289-300; | LPERGDDAGPKP; | aa305-323 |
| | aa304-312 | EGGDEGPQP | |
| 22B7 | aa286-321 | NAQLPERGDDAGPKPPEGEG | aa288-304, |
| | | GDEGPQPPQGGGEQDA | aa305-323 |
| 14D9 | aa289-324 | LPERGDDAGPKPPEGEGG | aa288-304, |
| | | DEGPQPPQGGGEQDAPEV | aa305-323 |
| 19F9 | aa289-294 | LPERGD | aa288-304 |
| 23F8 | aa292-300 | RGDDAGPKP | aa288-304 |
| 14D12 | aa292-300 | RGDDAGPKP | aa288-304 |
| 20F4 | aa307-315 | DEGPQPPQG | aa305-323 |
| 14A8 | aa310-315 | PQPPQG | aa305-323 |
| 14G11 | aa313-321 | PQGGGEQDA | aa305-323 |
| 25E3 | aa322-330 | PEVPPVAPA | aa40-374 |
| 18B2 | aa337-345 | VYDPGTHTL | aa40-374 |

### Example 3

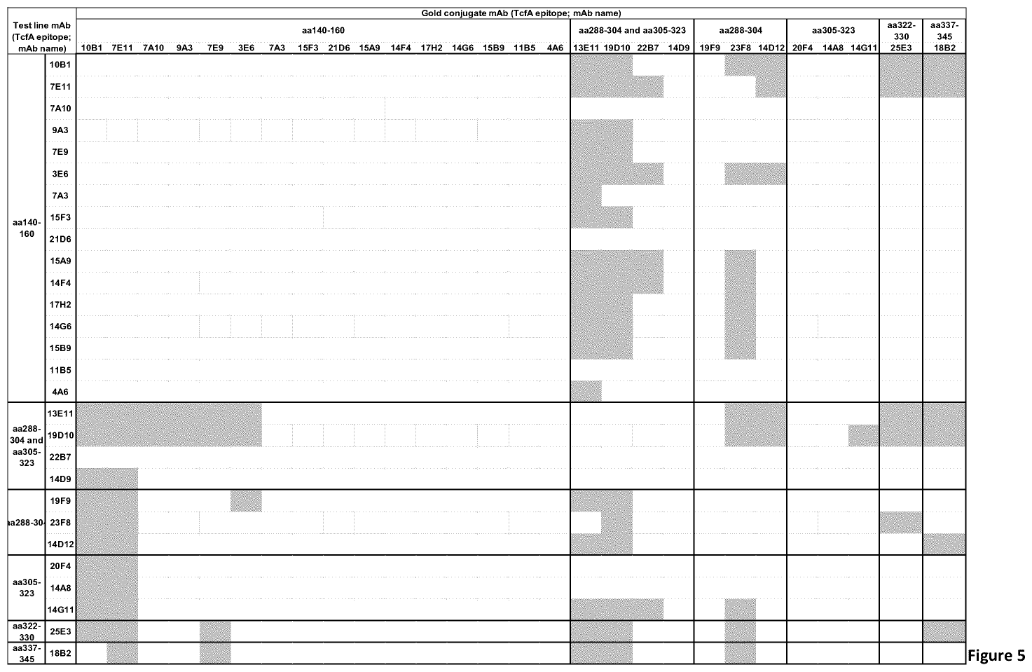

Anti-TcfA mAbs were evaluated for performance as a detector mAb (i.e., gold conjugate) and as a capture mAb (i.e., test line) in an LFI while holding all other LFI components constant (e.g., nitrocellulose, conjugate pad, sample pad, wicking pad, blocking buffers, chase buffer). A significant number of LFI mAb pair permutations were identified that effectively detected formaldehyde-inactivated B. pertussis cells (FIG. 5). Indeed, most of the ELISA-capable anti-TcfA mAbs evaluated by LFI formed at least 1 functional LFI pair (FIGS. 4 and 5). Notably, many anti-TcfA mAbs had comparable performance in ELISA against immobilized antigen (FIG. 4). However, in the LFI, some of the anti-TcfA mAbs worked far better than the others as test line mAbs, other anti-TcfA mAbs worked better as gold conjugate mAbs, and still other anti-TcfA mAbs worked well in both positions (e.g., anti-TcfA mAbs 14D9, 14D12, and 25E3, respectively) (FIG. 5). In a further analysis, the anti-TcfA mAbs were binned by their reactivity (or lack thereof) with the three TcfA peptides that were used to generate the polyclonal antibodies. It was found that anti-TcfA mAbs that target the same epitope failed to form functional mAb pairs for LFI (FIG. 5). However, when the anti-TcfA mAbs targeted different epitopes, functional mAb pairs for LFI were identified.

Several anti-TcfA mAb combinations for LFIs (FIG. 5) were advanced to BSL-2 testing to determine their cross-reactivity with a panel of ten other nasopharyngeal pathogens and to establish a preliminary limit of detection with viable B. pertussis cells. These LFIs were selected based on their strong signal with i) formaldehyde-inactivated B. pertussis cells (Tohama I strain), ii) detergent lysates of B. pertussis cells (Tohama I strain), and iii) conditioned media (not formaldehyde-treated) from a second strain of B. pertussis (strain 165) grown as liquid cultures in Stainer-Scholte medium. Three of the LFIs (14D12 test line+7E11 gold conjugate; 25E3 test line+10B1 gold conjugate; 14D9 test line+7E11 gold conjugate) were found to have no detectable cross-reactivity with viable cell suspensions in PBS of Haemophilus parainfluenzae, Haemophilus influenzae, Moraxella catarrhalis, Streptococcus pneumoniae, Streptococcus pyogenes, Corynebacterium diphtherias, Staphylococcus epidermidis, Bordetella parapertussis, Bordetella holmesii, or Bordetella bronchiseptica. One LFI had reactivity with H. influenza and B. holmesii. A different LFI had reactivity with S. epidermidis. One LFI (13E11 and 14D12 test line+10B1 gold conjugate) was subjected to additional cross-reactivity testing, in which cross-reactivity was evaluated with 40 other species of bacteria and fungi potentially found in nasopharyngeal specimens (Table 2). This expanded cross-reactivity testing of Table 2 was performed by testing LFIs in triplicate with samples containing 3.33×10⁷ CFU/mL of the indicated pathogen in extraction buffer.

TABLE 2
Pertussis LFI cross-reactivity testing (14D12 and
13E11 test line + 10B1 gold conjugate).

| | LFI |
|---|---|
| Microorganism (3.33 × 10⁷ CFU/mL) | Result |
| Acinetobacter baumanni | Negative |
| Acinetobacter calcoaceticus | Negative |
| Acinetocacter Iwoffii | Negative |
| Aspergillus fumigatus | Negative |
| Bacillus cereus | Negative |
| Bacillus subtilis | Negative |
| Bacteroides fragilis | Negative |
| Bordetella bronchiseptica (ATCC 19395) | Negative |
| Bordetella bronchiseptica (ATCC BAA-588) | Negative |
| Bordetella holmesii (F061) | Negative |
| Bordetella parapertussis (A747) | Negative |
| Bordetella parapertussis (C510) | Negative |
| Candida albicans | Negative |
| Candida glabrata | Negative |
| Citrobacter amalonaticus | Negative |
| Citrobacter freundii | Negative |
| Citrobacter koseri | Negative |
| Cornebacterium diphtheria | Negative |
| Enterobacter aerogenes | Negative |
| Enterobacter cloacae | Negative |
| Enterococcus faecalis (Z346) | Negative |
| Enterococcus faecium (Z265) | Negative |
| Escherichia coli (O157) | Negative |
| Haemophilus influenzae | Negative |
| Haemophilus parainfluenzae (ATCC33392) | Negative |
| Legionella pneumophila (Philadelphia) | Negative |
| Moraxella catarrhalis | Negative |
| Morganella morganii (Z098) | Negative |
| Mycoplasma pneumonia (M129) | Negative |
| Proteus mirabilis (Z050) | Negative |
| Proteus vulgaris (Z129) | Negative |
| Pseudomonas aeruginosa | Negative |
| Staphylococcus epidermidis | Negative |
| Stenotrophomonas maltophilia (Z074) | Negative |
| Streptococcus agalactiae (Z019) | Negative |
| Streptococcus dysgalactiae | Positive* |
| Streptococcus mitis | Negative |
| Streptococcus mutans (Z072) | Negative |
| Streptococcus pneumoniae | Negative |
| Streptococcus pyogenes | Negative |
| Streptococcus salivarius (Z127) | Negative |
| Streptococcus sanguinis (Z089) | Negative |

Testing for S. dysgalactiae was repeated by centrifuging cells to remove traces of growth medium and resuspending cells in extraction buffer at the same CFU/mL concentration; 3 of 3 LFI replicates were negative. S. dysgalactiae testing was also repeated by diluting cells. At a concentration of 8.3 × 10⁶ CFU/mL, 3 of 3 LFI replicates were negative.

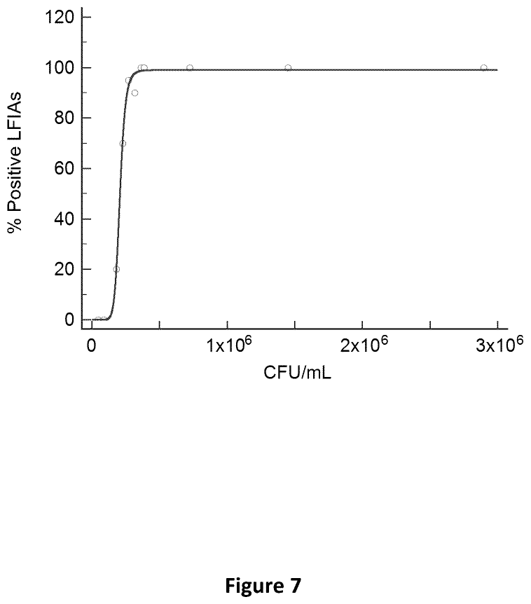

The preliminary limit of detection for five of the LFIs with viable B. pertussis cells was 2×10⁵ to 5×10⁵ CFU. Two of the three LFIs with no detectable cross-reactivity had preliminary limits of detection of 2×10⁵ CFU. These two LFIs are: 1) mAb 14D12 as the test line and mAb 7E11 as the gold conjugate; and 2) mAb 25E3 as the test line and mAb 10B1 as the gold conjugate. The sixth LFI (14D12 and 13E11 test line+10B1 gold conjugate) was subjected to detailed limit of detection testing and was determined to have an analytical sensitivity limit of 3×10⁵ CFU/mL (1.8×10⁴ CFU per LFI test) (FIG. 7). For comparison, the best pAb based LFI (see Example 1) had a final limit of detection of 1.6×10⁵ CFU. However, achieving that limit of detection with the pAb based LFI required i) heating the B. pertussis cells in 0.1% SDS at 65° C. for 1 hour, and ii) incorporating an 80 mm assay run length, which necessitated a longer assay run time (optimal run time was 30 minutes, though strong positives were visible sooner). The instant mAb based LFIs have better limit of detection despite their use of viable B. pertussis cells with only a simple 5 minute room temperature incubation in extraction buffer and short development times of 15-20 minutes due to a 60 mm assay run length, of which 25 mm is nitrocellulose.

B. pertussis is strictly a human pathogen that lacks an animal or environmental reservoir (Mattoo, et al., Clin. Microbiol. Rev., (2005) 18(2):326-82). However, a baboon model has been developed that is the only animal model that accurately reproduces the full disease course seen in human infection (Warfel, et al., Expert Rev. Vaccines (2014) 13(10):1241-52; Merkel, et al., J. Infect. Dis. (2014) 209 Suppl 1:S20-3; Warfel, et al., Infect. Immun. (2012) 80(4):1530-6; Warfel, et al., Proc. Natl. Acad. Sci. (2014) 111(2):787-92). This well-established, clinically relevant model of naturally transmitted infection in baboons can be used to provide nasopharynx samples to study the ability of the LFIs to diagnose various disease stages.

### Example 4

A library of B. pertussis mAbs has been generated. The mAbs bind viable and formaldehyde-inactivated B. pertussis cells. In addition to use as diagnostic reagents, these mAbs are valuable as therapeutics, prophylactics, and/or research tools (e.g., for fluorescence microscopy on fresh or fixed tissue sections, etc.).

A mAb-based LFI was optimized for sensitive, specific detection of B. pertussis. The LFI comprised 14D12 and 13E11 in the test line and 10B1 as the conjugated antibody. The LFI detected B. pertussis antigen in ≤20 minutes with a simple protocol, enabling diagnostic use at the point-of-care.

Human infant nasopharyngeal washes contain 7×10⁷ to 8×10¹⁰ CFU/mL (Eby, et al., Infect. Immun. (2013) 81(5):1390-8; Tenenbaum, et al., Eur. J. Clin. Microbiol. Infect. Dis. (2012) 31(11):3173-82). These CFU/ml values are approximately 200-fold to 260,000-fold greater than the LFI's analytical sensitivity limit (i.e., C95). Indeed, as seen in FIG. 7, the LFI has an estimated analytical sensitivity (C95) of 3×10⁵ CFU/mL (1.8×10⁴ CFU per LFI test) for viable B. pertussis (Tohama I) in extraction buffer. Nonlinear regression analysis using a 4-parameter logistic model (MedCalc Software) was used to determine the line of best fit. The equation for the line was used to calculate the concentration at which 95% of LFIs would be positive. Each concentration was evaluated by 20 LFI replicates; all LFIs were from one lot.

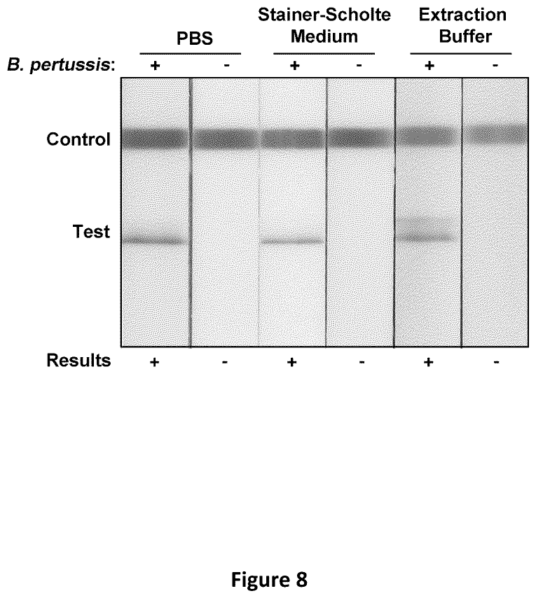

The LFI produced strong signal with multiple B. pertussis strains and sample types. As an example, FIG. 8 shows formaldehyde-inactivated B. pertussis cells (Tohama I strain) cells in PBS (OD₆₀₀ of 0.1) vs. PBS alone; clarified, 0.2 μm-filtered supernatant from Stainer-Scholte B. pertussis (strain 165) cultures vs. uninoculated Stainer-Scholte medium (both diluted 1:128 in PBS); and formaldehyde-inactivated B. pertussis cells (Tohama I strain) in PBS (OD₆₀₀ of 0.1) lysed for 5 minutes in extraction buffer vs. extraction buffer alone. The LFI has shown reactivity with at least 9 different strains of B. pertussis including: Tohama I, 165, D420, H973, H792,

H735, E431, A639, and CNCTC Hp 12/63 [623].

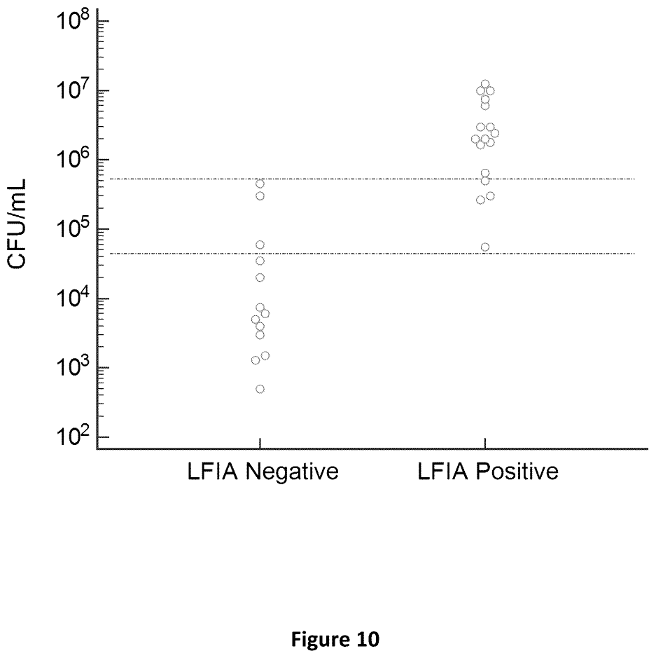

Nasopharyngeal washes from baboons infected by natural, airborne transmission have greater than or equal to 5×10⁵ CFU/mL from approximately Day 15 to Day 33 post-exposure (Warfel, et al., Proc. Natl. Acad. Sci. (2014) 111(2):787-92). The LFI detected antigen from infected baboon nasopharyngeal washes containing as little as 5.5×10⁴ CFU/mL (3.3×10³ CFU per LFI test) (FIG. 10). The LFI consistently reported positive results with infected baboon nasopharyngeal washes greater than or equal to 5×10⁵ CFU/mL (3×10⁴ CFU per LFI test) (FIG. 10). No LFI false-positives were observed.

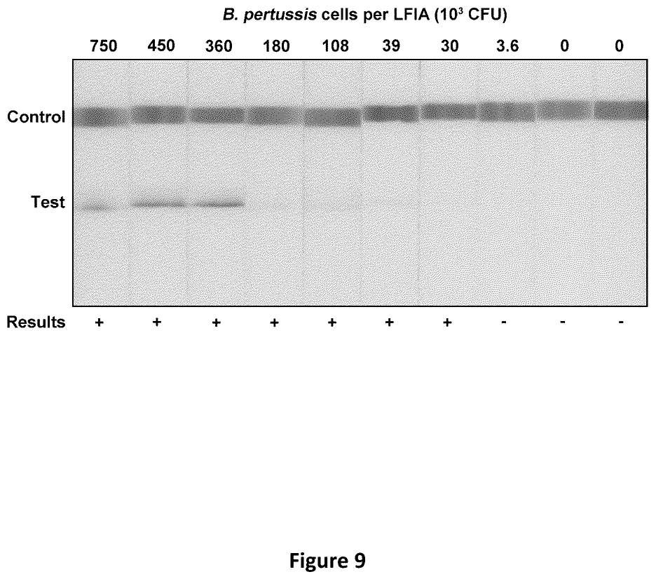

Specifically, as seen in FIGS. 9 and 10, the LFI reacted with nasopharyngeal washes from baboons infected with B. pertussis (strain D420). Individual nasopharyngeal washes from baboons challenged with B. pertussis and containing the listed CFU per LFI testing volume were incubated with extraction buffer for 5 minutes at room temperature. LFI development time was 15 minutes.

As seen in FIG. 10, all infected baboon nasopharyngeal washes with greater than or equal to 5×10⁵ CFU/mL were positive by LFI (top horizontal reference line), and all infected baboon nasopharyngeal washes with less than or equal to 3.5×10⁴ CFU/mL were negative by LFI (bottom horizontal reference line). A total of 30 nasopharyngeal washes from baboons challenged with B. pertussis were analyzed by LFI. The LFI produced no false-positives with 11 baboon NP washes that had 0 CFU/mL (i.e. the LFI had 100% specificity).

In addition, the LFI has been tested with human patient samples (nasopharyngeal swabs). The LFI was negative with 2 of 2 patient samples that were diagnosed by RT-PCR as negative for pertussis. The LFI was positive with 1 patient sample that was diagnosed by RT-PCR as being positive for pertussis and with a high bacterial burden. The LFI was negative with 1 patient sample that was diagnosed by RT-PCR as positive for pertussis but with a significantly lower bacterial burden. The 1 positive LFI with the 1 patient who was positive by RT-PCR shows proof of concept for detection of B. pertussis in human nasopharyngeal swab specimens.

Together, the baboon and human results indicate that the LFI can detect infection from two different types of nasopharyngeal specimens: nasopharyngeal washes and nasopharyngeal swabs.

The mAb-based LFI for detection of B. pertussis antigen allows for early, point-of-care diagnosis of pertussis using minimally-invasive nasopharyngeal specimens. The availability of a rapid and simple test for detection of pertussis will increase early diagnosis, as well as facilitate immediate triage, treatment, and outbreak containment.

Several publications and patent documents are cited in the foregoing specification in order to more fully describe the state of the art to which this invention pertains. The disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated antibody or antigen binding fragment thereof immunologically specific for tracheal colonization factor A (TcfA), wherein said antibody or fragment thereof specifically binds amino acids 140-160, amino acids 229-240, amino acids 288-304, amino acids 286-321, amino acids 289-324, amino acids 305-323, amino acids 322-330, or amino acids 337-345 of TcfA.

2. The antibody or fragment thereof of claim 1, wherein said antibody or fragment thereof specifically binds amino acids 140-150, amino acids 148-159, amino acids 151-156, amino acids 151-159, amino acids 229-240, amino acids 289-300, amino acids 305-312, amino acids 286-321, amino acids 289-324, amino acids 289-294, amino acids 292-300, amino acids 307-315, amino acids 310-315, amino acids 313-321, amino acids 322-330, or amino acids 337-345 of TcfA.

3. The antibody or fragment thereof of claim 1, wherein said antibody or fragment thereof comprises at least one complementarity determining region from 14D12, 23F8, 18B2, 20F4, 14G11, 13E11, 10B1, 7E11, or 3E6.

4. The antibody or fragment thereof of claim 1, wherein said antibody or fragment thereof comprises all six complementarity determining regions from 14D12, 23F8, 18B2, 20F4, 14G11, 13E11, 10B1, 7E11, or 3E6.

5. The antibody or fragment thereof of claim 1, wherein said antibody or fragment thereof comprises
a) a heavy chain comprising NYAMS (SEQ ID NO: 24), SISSGGSYIYYSDSVKG (SEQ ID NO: 25), and GAHGNFDY (SEQ ID NO: 26) and/or a light chain comprising RTSETVDYDGDSYMN (SEQ ID NO: 27), GASNVES (SEQ ID NO: 28), and QQNRKLPYT (SEQ ID NO: 29);
b) a heavy chain comprising DYGMH (SEQ ID NO: 36), YISSGSRTIYYADTVKG (SEQ ID NO: 37), and LGYGYDWYFDV (SEQ ID NO: 38) and/or a light chain comprising KSSQSLLDSDGRTYLN (SEQ ID NO: 39), LVSKLDS (SEQ ID NO: 40), and WQGTHFPQT (SEQ ID NO: 41);
c) a heavy chain comprising NYWIG (SEQ ID NO: 48), DIYPGGVYTNYNENFKG (SEQ ID NO: 49), and GGKYGNFFAMDY (SEQ ID NO: 50) and/or a light chain comprising RSSKSLLYKDGKTYLN (SEQ ID NO: 51), LMSTRAS (SEQ ID NO: 52), and QQLVEYPFT (SEQ ID NO: 53);
d) a heavy chain comprising NYGMN (SEQ ID NO: 60), WINTYTGEPTYADDFKG (SEQ ID NO: 61), and AATGYFDY (SEQ ID NO: 62) and/or a light chain comprising KSSQSLLYSSNQKNYLA (SEQ ID NO: 63), WASTRES (SEQ ID NO: 64), and QQYYNEYT (SEQ ID NO: 65);
e) a heavy chain comprising DYYMS (SEQ ID NO: 72), FIRNKANGYTTEYSASVKG (SEQ ID NO: 73), and YRRDYYGSLNYYTMDY (SEQ ID NO: 74) and/or a light chain comprising RASENIYSYLA (SEQ ID NO: 75), NAKTLAE (SEQ ID NO: 76), and QNHYGIPLT (SEQ ID NO: 77);
f) a heavy chain comprising DYGMH (SEQ ID NO: 84), YISSGSSTIYYADTVKG (SEQ ID NO: 85), and PRSGRYFDY (SEQ ID NO: 86) and/or a light chain comprising KSSQSLLDSNGNTYLH (SEQ ID NO: 87), LVSKLDS (SEQ ID NO: 88), and LQGTHFPYT (SEQ ID NO: 89);
g) a heavy chain comprising DTYIH (SEQ ID NO: 96), RIDPANGNTIYASKFQG (SEQ ID NO: 97), and MDY and/or a light chain comprising KSSQSLLHSNGKTYLN (SEQ ID NO: 99), LVSKLDS (SEQ ID NO: 100), and LQATHFPHT (SEQ ID NO: 101);
h) a heavy chain comprising DTYIH (SEQ ID NO: 108), RIDPANGNIIYASKFQG (SEQ ID NO: 109), and MDY and/or a light chain comprising KSSQSLLHSNGKTYLN (SEQ ID NO: 111), LVSKLDS (SEQ ID NO: 112), and LQATHFPHT (SEQ ID NO: 113); or
i) a heavy chain comprising DYYMS (SEQ ID NO: 120), FIRNKAKGYTTDYSASVKG (SEQ ID NO: 121), and NYDYSMDY (SEQ ID NO: 122) and/or a light chain comprising RASDNIHKYLA (SEQ ID NO: 123), NAKTLAD (SEQ ID NO: 124), and QHFWSTPLT (SEQ ID NO: 125).

6. The antibody or fragment thereof of claim 1, wherein said antibody or fragment thereof comprises
a) a heavy chain comprising:
(SEQ ID NO: 22)
EVMLVESGGALVKPGGSLKLSCAASGITFSNYAMSWIRQTPEKRLEWVASI
SSGGSYIYYSDSVKGRFTISRDNARNTLNLQMSSLRSEDTAMYYCVRGAHG
NFDYWGQGTTLTVSS
and/or a light chain comprising:
(SEQ ID NO: 23)
DIVLTQSPASLAVSLGQRATISCRTSETVDYDGDSYMNWYQQKSGQPPKLL
ISGASNVESGVPARFSGSGSGTDFSLNIHPVEEDDITMYFCQQNRKLPYTF
GSGTKLEMK;
b) a heavy chain comprising:
(SEQ ID NO: 34)
EVOLVESGGGLVKPGGSRKLSCAASGFTFSDYGMHWVRQAPEKGLEWVAYI
SSGSRTIYYADTVKGRFTISRDNAKNTLFLQMTSLRSEDTAMYYCARLGYG
YDWYFDVWGTGTTVTVSS
and/or a light chain comprising:
(SEQ ID NO: 35)
DVVMTQTPLTLSVTIGQPASISCKSSQSLLDSDGRTYLNWLLQRPGQSPKR
LIYLVSKLDSGVPDRFTGSGSGTDFTLKISRVEAEDLGIYYCWQGTHFPQT
FGGGTKLEIK;
c) a heavy chain comprising:
(SEQ ID NO: 46)
QVQLQQSGAELVRPGTSVKMSCKAAGYTFTNYWIGWVKQRPGHGLEWIGDI
YPGGVYTNYNENFKGKATLTADTSSSTAHMQLSSLTSEDSAIYYCVRGGKY
GNFFAMDYWGQGTSVTVSS
and/or a light chain comprising:
(SEQ ID NO: 47)
DIVITQDELSNPVTSGESVSISCRSSKSLLYKDGKTYLNWFLQRPGQSPQL
LIYLMSTRASGVSDRFSGSGSGTDFTLEISRVKAEDVGVYYCQQLVEYPFT
FGSGTKLEIK;
d) a heavy chain comprising:
(SEQ ID NO: 58)
QIQLVQSGPELKKPGETVKISCKASGYTFTNYGMNWVKQAPGKGLKWMGWI
NTYTGEPTYADDFKGRFAFSLETSATTAYLQINNLKNEDTATYFCARAATG
YFDYWGQGTTLTVSS
and/or a light chain comprising:
(SEQ ID NO: 59)
DIVMSQSPSSLAVSAGEKVTMSCKSSQSLLYSSNQKNYLAWYQQKPGQSPK
LLIYWASTRESGVPDRFTGSGSGTDFTLTISSVKAEDLAVYYCQQYYNEYT
FGGGTKLEIK;
e) a heavy chain comprising:
(SEQ ID NO: 70)
EVKLVESGGGLVQPGGSLSLSCAASGFTFTDYYMSWVRQPPGKALEWLGFI
RNKANGYTTEYSASVKGRFTISRDNSQSILYLQMNALRAEDSATYYCARYR
RDYYGSLNYYTMD YWGQGTSVTVSS
and/or a light chain comprising:
(SEQ ID NO: 71)
DIQMTQSPASLSASVGETVTITCRASENIYSYLAWYQQKQGKSPQLLVYNA
KTLAEGVPSRFSGSGSGTQFSLKINSLQPEDFGSYYCQNHYGIPLTFGAGT
KLELK;
f) a heavy chain comprising:
(SEQ ID NO: 82)
EVOLVESGGGLVKPGGSRKLSCAASGFTFSDYGMHWVRQAPEKGLEWVAYI
SSGSSTIYYADTVKGRFTISRDNAKNTLFLQMTSLRSEDTAMYYCARPRSG
RYFDYWGQGTTLTVSS
and/or a light chain comprising:
(SEQ ID NO: 83)
DVMMTQTPLTLSVTIGQPASISCKSSQSLLDSNGNTYLHWLLQRPGQSPKI
LIYLVSKLDSGVPDRFSGSGSGTDFTLKISRVEAEDLGVYYCLQGTHFPYT
FGGGTKLEIK;
g) a heavy chain comprising:
(SEQ ID NO: 94)
EVQLQQSGAELVKPGASVKLSCTASGFNIKDTYIHWVKQRPEQGLEWIGRI
DPANGNTIYASKFQGKAPITAVTSSNTAYMQFSSLTSGDTAVYYCTAMDYW
GQGTSVTVSS
and/or a light chain comprising:
(SEQ ID NO: 95)
DVVMTQTPLTLSVTIGQPASISCKSSQSLLHSNGKTYLNWLLQRPGQSPKL
LIYLVSKLDSGVPDRFSGSGSGTDFTLKISRVEAEDLGVYYCLQATHFPHT
FGSGTKLEIK;
h) a heavy chain comprising:
(SEQ ID NO: 106)
EVQLQQSGAELVKPGASVKLSCTASGFNIKDTYIHWVKQRPEQGLEWIGRI
DPANGNIIYASKFQGEATITADTSSNTAYMQLSSLTSGDTAVYYCSAMDYW
GQGTSVTVSS
and/or a light chain comprising:
(SEQ ID NO: 107)
DVVMTQTPLTLSLTIGQPASISCKSSQSLLHSNGKTYLNWLLQRPGQSPKL
LIYLVSKLDSGVPDRFSGSGSGTDFTLKISRVEAEDLGVYYCLQATHFPHT
FGSGTKLEIK;
or
i) a heavy chain comprising:
(SEQ ID NO: 118)
EVKLVESGGGLVQPGGSLRLSCATSGFTFTDYYMSWVRQPPGKALEWMGFI
RNKAKGYTTDYSASVKGRFTISRDDSQSILYLQMNTLRPEDSATYYCARNY
DYSMDYWGQGTSVTVSS
and/or a light chain comprising:
(SEQ ID NO: 119)
DIQLTQSPASLSASVGETVTITCRASDNIHKYLAWYQQKQGKSPQRLVYNA
KTLADGVPSRFNGSGSGTQYSLKINSLQPEDFGIYYCQHFWSTPLTFGAGT
KLELK.

7. The antibody or fragment thereof of any one of claims 1-6 conjugated to at least one detectable agent.

8. The antibody or fragment thereof of claim 7, wherein said detectable agent is a gold nanoparticle.

9. A composition comprising an antibody or fragment thereof of any one of claims 1-8 and a carrier.

10. A method of detecting Bordetella pertussis in a sample, said method comprising contacting the sample with at least one antibody or fragment thereof of any one of claims 1-8.

11. The method of claim 10, wherein said sample is a biological sample obtained from a subject.

12. The method of claim 11, wherein said biological sample is a nasopharyngeal swab, aspirate, or wash.

13. A methods for inhibiting, treating, and/or preventing of pertussis and/or a B. pertussis infection in a subject in need thereof, said method comprising administering an antibody or fragment thereof of any one of claims 1-8 to the subject.

14. An immunoassay comprising at least one antibody or fragment thereof of any one of claims 1-8.

15. The immunoassay of claim 14, wherein said immunoassay is a lateral flow immunoassay test strip, wherein said lateral flow immunoassay test strip comprises a test site comprising one or more anti-TcfA antibodies and a conjugation pad comprising one or more anti-TcfA antibodies conjugated to a detectable agent.

16. The immunoassay of claim 15, wherein the conjugated antibody specifically binds amino acids 139-150 or amino acids 151-156 of TcfA.

17. The immunoassay of claim 16, wherein the conjugated antibody specifically binds amino acids 139-150 of TcfA.

18. The immunoassay of any one of claims 15-17, wherein the test site antibody specifically binds amino acids 289-324 of TcfA.

19. The immunoassay of any one of claims 15-17, wherein the test site antibody specifically binds amino acids 289-294 of TcfA.

20. The immunoassay of any one of claims 15-17, wherein the test site antibody specifically binds amino acids 292-300 of TcfA.

21. The immunoassay of any one of claims 15-17, wherein the test site antibody specifically binds amino acids 322-330 of TcfA.

22. The immunoassay of claim 15, wherein the conjugated antibody specifically binds amino acids 289-324 of TcfA.

23. The immunoassay of claim 22, wherein the test site antibody specifically binds amino acids 139-150 of TcfA.

24. The immunoassay of claim 15, wherein the conjugated antibody specifically binds the same epitope as 10B1 and said test site comprises antibodies which bind the same epitope as 14D12 and antibodies which bind the same epitope as 13E11.

25. A method of detecting Bordetella pertussis in a sample, said method comprising analyzing the sample with an immunoassay of any one of claims 14-24.
